(19) United States
(12) Patent Application Publication
(10) Pub. No.: US 2025/0207134 A1
(43) Pub. Date: Jun. 26, 2025

(54) Use of Non-Coding Nucleic Acid for Crop Improvement and Protection Against Microbes

(71) Applicant: University of Kentucky Research Foundation, Lexington, KY (US)

(72) Inventors: Pradeep Kachroo, Lexington, KY (US); Aardra Kachroo, Lexington, KY (US); Gah-Hyun Lim, Lexington, KY (US); Shine Baby, Lexington, KY (US)

(21) Appl. No.: 19/015,426

(22) Filed: Jan. 9, 2025

Related U.S. Application Data
(62) Division of application No. 17/327,631, filed on May 21, 2021.
(60) Provisional application No. 63/028,376, filed on May 21, 2020.

(51) Int. Cl. C12N 15/113; A01N 57/16

(52) U.S. Cl. CPC C12N 15/113; A01N 57/16; C12N 2310/14

(57) ABSTRACT

A compound and method for conferring systemic acquired resistance (SAR) in plants are provided. The compound includes a nucleotide sequence derived from trans-acting small interfering RNA3a (TAS3a). The method includes exogenously applying a compound having a nucleotide sequence derived from trans-acting small interfering RNA3a (TAS3a).

### RELATED APPLICATIONS

This application is a division of U.S. patent application Ser. No. 17/327,631, filed May 21, 2021, which claims the benefit of U.S. Provisional Application Ser. No. 63/028,376, filed May 21, 2020, the entire disclosure of which is incorporated herein by this reference.

### GOVERNMENT INTEREST

This invention was made with government support under grant number 051909 awarded by the National Science Foundation (NSF). The government has certain rights in the invention.

### SEQUENCE LISTING

The contents of the electronic sequence listing (Kachroo. 13177N.2314US2.xml; Size: 15,011 bytes; and Date of Creation: Jan. 8, 2025) is herein incorporated by reference in its entirety.

### TECHNICAL FIELD

The present disclosure is directed to compounds and methods for protecting crops against microbes. In particular, the disclosure is directed to non-coding nucleic acids and the use thereof for crop improvement and protection against microbes.

### BACKGROUND

Pathogen infection can result in the induction of sophisticated signal transduction pathways in the local infected tissues, which are generally categorized as basal or pathogen-associated molecular patterns-triggered immunity (PTI), and race-specific or effector-triggered immunity (ETI). PTI is induced when the extracellular pattern-recognition receptors in the plant recognize conserved pathogen-derived molecules termed elicitors. ETI is induced when plant resistance (R) proteins recognize specialized pathogen effectors termed avirulence (avr) factors.

In addition to these local responses, plants can also induce systemic resistance particularly in response to the induction of ETI. This form of resistance, commonly referred to as systemic acquired resistance (SAR), is a type of broad-spectrum resistance mechanism in plants. SAR often leads to resistance at the whole plant level and involves the local generation of signal(s) at the primary infection site followed by their systemic transport throughout the plant. These signals then arm the distal uninfected portions against subsequent secondary infections. Its indisputable advantage for managing crop diseases makes SAR one of the intensely studied topics in plant biology. The last decade has witnessed several breakthroughs in the SAR field, resulting in the elucidation of many crucial aspects of SAR signaling. However, even though first identified as a form of plant immunity nearly 100 years ago, the identity of the mobile signal(s) conferring SAR remain unknown. Potentially, the identification of SAR mobile signal(s) and the knowledge of their dynamic movement could greatly facilitate the application of SAR.

Accordingly, there remains a need for compounds and methods to confer SAR in plants.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a compound for conferring systemic acquired resistance (SAR) in plants, the compound including a nucleotide sequence derived from trans-acting small interfering RNA3a (TAS3a). In some embodiments, the compound includes an RNA transcript including a sequence according to SEQ ID NO: 2, wherein the RNA transcript includes at least one mutation or modification to the sequence thereof. In some embodiments, the modification includes a ribose 2′/3′-ribose modification, a 3′-end modification, a locked nucleic acids (LNA) modification, conjugation of a nanoparticle (NP), or a combination thereof. In some embodiments, the 2′-ribose modification includes 2′-fluorination, 2′-oxymethilation, 2′amination of pyrimidines, or a combination thereof. In some embodiments, the 3′-end modification includes replacing the 3′-end phosphate group with phosphotioate or boranophosphate.

In some embodiments, the compound includes an RNA transcript including a sequence according to SEQ ID NO: 3 or SEQ ID NO: 4, wherein the RNA transcript includes at least one mutation or modification to the sequence thereof. In some embodiments, the modification includes a ribose 2′/3′-ribose modification, a 3′-end modification, a locked nucleic acids (LNA) modification, conjugation of a nanoparticle (NP), or a combination thereof. In some embodiments, the 2′-ribose modification includes 2′-fluorination, 2′-oxymethilation, 2′amination of pyrimidines, or a combination thereof. In some embodiments, the 3′-end modification includes replacing the 3′-end phosphate group with phosphotioate or boranophosphate.

In some embodiments, the compound includes an RNA transcript including a sequence according to SEQ ID NO: 6 or SEQ ID NO: 8, wherein the RNA transcript includes at least one mutation or modification to the sequence thereof. In some embodiments, the modification includes a ribose 2′/3′-ribose modification, a 3′-end modification, a locked nucleic acids (LNA) modification, conjugation of a nanoparticle (NP), or a combination thereof. In some embodiments, the 2′-ribose modification includes 2′-fluorination, 2′-oxymethilation, 2′amination of pyrimidines, or a combination thereof. In some embodiments, the 3′-end modification includes replacing the 3′-end phosphate group with phosphotioate or boranophosphate.

In some embodiments, the compound includes an RNA transcript including a sequence according to SEQ ID NO: 10, wherein the RNA transcript includes at least one mutation or modification to the sequence thereof. In some embodiments, the modification includes a ribose 2′/3′-ribose modification, a 3′-end modification, a locked nucleic acids (LNA) modification, conjugation of a nanoparticle (NP), or a combination thereof. In some embodiments, the 2′-ribose modification includes 2′-fluorination, 2′-oxymethilation, 2′amination of pyrimidines, or a combination thereof. In some embodiments, the 3′-end modification includes replacing the 3′-end phosphate group with phosphotioate or boranophosphate.

Also provided herein, in some embodiments, is a method of conferring systemic acquired resistance (SAR) in plants, the method including exogenously applying a compound having a nucleotide sequence derived from trans-acting small interfering RNA3a (TAS3a). In some embodiments, the compound includes a sequence according to any of SEQ ID NOs: 1-10, mutations thereof, or modifications thereof. In some embodiments, the modifications thereof include a ribose 2′/3′-ribose modification, a 3′-end modification, a locked nucleic acids (LNA) modification, conjugation of a nanoparticle (NP), or a combination thereof.

Further features and advantages of the presently-disclosed subject matter will become evident to those of ordinary skill in the art after a study of the description, figures, and non-limiting examples in this document.

### BRIEF DESCRIPTION OF THE DRAWINGS

FIGS. 1A-H show graphs illustrating that a mutation in ago1 or ago7 or overexpression of DRB2 compromises SAR. (A-B) SAR response in distal leaves of (A) 35S-DRB and (B) ago plants treated locally with MgCl₂ or avrRpt2. All transgenic and mutant plants were in Col-0 background. The virulent pathogen (DC3000) was inoculated 48 h post-local treatments. CFU indicates colony forming units. Asterisks denote a significant difference with respective mock-inoculated samples (t test, P<0.0001). The experiment was repeated six times with similar results for all but ago1-27 mutant which showed partial SAR in two repeats. (C) Real-time quantitative RT-PCR showing relative expression levels of PR-1 in mock- and avrRpt2-inoculated Col-0, ago1 and ago7 plants at 24 h post inoculation. The error bars indicate SD (n=3). Results are representative of two independent experiments. Asterisks denote a significant difference between respective mock- and avr-inoculated samples (t test, P<0.0001). (D) SA (left panel) and SAG (right panel) levels in local tissues after mock (10 mM MgCl₂)- and pathogen (avrRpt2)-inoculations. The leaves were sampled 48 h post treatments and the experiment was repeated two times with similar results. Asterisks denote a significant difference with respective mock-inoculated samples (t test, P<0.0001). (E) Pip, (F) AzA, and (G) G3P levels in local tissues of Col-0, ago1 and ago7 plants after mock- and avrRpt2-inoculations. The leaves were sampled (E-F) 48 h or (G) 24 h post treatments. Asterisks denote a significant difference with mock (t test, P<0.0001). These experiments were repeated three times with similar results. (H) SAR response in distal leaves of Col-0, ago1 or ago7 plants treated locally with water, SA (500 μM), Pip (1000 μM), AzA (1000 μM), or G3P (100 μM). The virulent pathogen (DC3000) was inoculated 48 h post-local treatments. Asterisks denote a significant difference with mock (t test, P<0.0005). The experiment was repeated three times with similar results.

FIGS. 2A-N show an image and graphs illustrating that a mutation in TAS3a compromises SAR in SA-, Pip-, G3P- and AzA-independent manner. (A) Morphological phenotypes of four-week-old Col-0, tas3a and tas3b plants. Only tas3a plants showed characteristic zippy phenotype. (B) SAR response in distal leaves of tas plants treated locally with MgCl2 or avrRpt2. All the tas mutants were in Col-0 background. The virulent pathogen (DC3000) was inoculated 48 h post-local treatments. CFU indicates colony forming units. Asterisks denote a significant difference with respective mock-inoculated samples (t test, P<0.0001). (C) SA (left panel) and SAG (right panel) levels in local tissues of Col-0 and tas3a plants after mock (10 mM MgCl2)- and pathogen (avrRpt2)-inoculations. The leaves were sampled 48 h post treatments and the experiment was repeated two times with similar results. Asterisks denote a significant difference with respective mock-inoculated samples (t test, P<0.0001). (D-E) Pip levels in (D) local or (E) distal tissues of Col-0 and tas3a plants after mock- and avrRpt2-inoculations. The leaves were sampled 48 h post treatments. Asterisks denote a significant difference with mock (t-test, P<0.0001). The experiment was repeated three times with similar results. (F) G3P levels in local tissues of Col-0 and tas3a plants after mock- and avrRpt2-inoculations. The leaves were sampled 24 h post treatments. Asterisks denote a significant difference with mock (t test, P<0.0001). This experiment was repeated three times with similar results. (G) AzA, (H) G3P, and (I) SA levels in PEX collected from mock (PEXMgCl2)- and avrRpt2 (PEXavrRpt2)-inoculated plants. Results are representative of four independent experiments. Single (t test, P<0.0001) and double (t test, P<0.004) asterisks denote a significant difference with respective mock-inoculated samples or between indicated pairs, respectively. (J-M) SAR response in distal leaves of Col-0, and tas3a plants treated locally with water or (J) SA (500 μM), (K) Pip (1000 μM), (L) AzA (1000 μM), or (M) G3P (100 μM). The virulent pathogen (DC3000) was inoculated 48 h post-local treatments. Asterisks denote a significant difference with mock (t test, P<0.0001). The experiment was repeated three times with similar results. (N) Venn diagrams showing overlap between the number of genes induced or repressed in local and distal tissues of Col-0 and tas3a plants after inoculation with avrRpt2.

FIGS. 3A-M show graphs and images illustrating that TAS3a and Tasi-ARFs confer robust SAR. (A) SAR response in distal leaves of Col-0 plants treated locally with avrRpt2 or TAS3a 153 or 555 nt transcripts (0.039 nmol/ml). The virulent pathogen (DC3000) was inoculated 48 h post-local treatments. Asterisks denote a significant difference with mock (t test, P<0.0001). The experiment was repeated five times with similar results. (B-C) SAR response in distal leaves of Col-0 and tas3a plants treated locally with MgCl2, avrRpt2, or indicated TAS3a transcripts. The virulent pathogen (DC3000) was inoculated 48 h post-local treatments. CFU indicates colony forming units. Asterisks denote a significant difference with respective mock-inoculated samples (t test, P<0.0001). (D) SAR response in distal leaves of Col-0 plants treated locally with MgCl2, avrRpt2 or wild-type or mutant 555 nt TAS3a transcripts lacking 5′ and/or 3′ cleavage sites (see FIG. 9A). The virulent pathogen (DC3000) was inoculated 48 h post-local treatments. Asterisks denote a significant difference with respective mock-inoculated samples (t test, P<0.0001). (E) SAR response in distal leaves of Col-0 and miR390a plants treated locally with avrRpt2. The virulent pathogen (DC3000) was inoculated 48 h post-local treatments. Asterisks denote a significant difference with mock (t test, P<0.0001). The experiment was repeated three times with similar results. (F) Protein immunoblot showing levels of 5-6 kd protein encoded by the TAS3a ORF (see FIG. 9A) in Col-0 and tas3a plants. Ponceau-S staining of the immunoblot was used as the loading control. The experiment was repeated five times and the TAS3a specific protein was detected three times. (G-H) SAR response in distal leaves of Col-0 plants treated locally with avrRpt2, (G) TAS3a (153 nt) or (H) TAS3a (555 nt) transcripts containing AUG or AUU (T-153^{M}/T-555^{M}) start codon. The virulent pathogen (DC3000) was inoculated 48 h post-local treatments. Asterisks denote a significant difference with mock (t test, P<0.0001). The *a denotes significant difference between T-153^{M} and avr/wild-type transcript induced SAR. The experiment was repeated five (G) or two (H) times with similar results. (I-J) Real-time quantitative stem-loop RT-PCR showing relative expression levels of Tasi-ARFs D7 and D8 in Col-0 plants treated with (I) TAS3a (555 nt) transcript or (J) avrRpt2. The error bars indicate SD (n=3). Results are representative of two independent experiments. Asterisks denote a significant difference between mock (MgCl₂) and transcript treated samples (t test, P<0.005). (K) SAR response in distal leaves of Col-0 plants treated locally with MgCl₂ or Tasi-ARFs D7 or D8 (1 μM). The virulent pathogen (DC3000) was inoculated 48 h post-local treatments. Asterisks denote a significant difference with mock (t test, P<0.0001). The experiment was repeated three times with similar results. (L) Real-time quantitative stem-loop RT-PCR showing relative expression levels of Tasi-ARFs D7 and D8 in Col-0 and ago7 plants treated with avrRpt2. The error bars indicate SD (n=3). Results are representative of two independent experiments. Asterisks denote a significant difference between 0 and 3 h post treatment (t test, P<0.005). (M) SAR response in distal leaves of Col-0, ago7, or tas3a plants treated locally with MgCl₂, Tas3a₅₅₅ or Tasi-ARFs D7 or D8. The virulent pathogen (DC3000) was inoculated 48 h post-local treatments. Asterisks denote a significant difference with mock (t test, P<0.0001). The experiment was repeated three times with similar results.

FIGS. 4A-U show graphs and images illustrating that TAS3a confers SAR in ARF-dependent manner. (A) Real-time quantitative stem-loop RT-PCR showing relative expression levels of Tasi-ARFs D7 and D8 in Col-0 plants inoculated with buffer (MgCl₂) or avrRpt2. The error bars indicate SD (n=3). Results are representative of two independent experiments. NS indicates data not significantly different. (B-C) Real-time quantitative RT-PCR showing relative expression levels of TAS3a in PEX collected from (B) mock- and avrRpt2-inoculated Col-0 leaves or (C) inoculated leaves. The error bars indicate SD (n=4). Results are representative of four independent experiments. Asterisks denote a significant difference between samples harvested before (0 time) or indicated h post avrRpt2 inoculation (t test, P<0.001). (D) SAR response in Col-0 plants infiltrated with petiole exudates (PEX) collected from Col-0 that were treated either with MgCl₂ (PEX_{MgCl2}) or avrRpt2 (PEX_{avrRpt2}). One set of PEX was treated with 100 μM RNAase for one hour prior to infiltration into local leaves. The distal leaves were inoculated with virulent pathogen at 48 h post infiltration of primary leaves. Asterisks denote a significant difference with mock (t test, P<0.0001). The experiment was repeated three times with similar results. (E) Whole plant autoradiogram showing transport of wild-type TAS3a 153 or 555 nt transcripts to distal leaves of wild-type Col-0. Local leaves were co-infiltrated with 32p-ATP labeled TAS3a and 6 h post treatment the inoculated leaves were removed and the remaining plants were autoradiographed. The T-153^{M} represents mutant TAS3a transcript where the start codon AUG was replaced with AUU. (F) Percentage of ³²p-TAS3a associated radiolabel detected in the distal tissues of Col-0 plants infiltrated with 142.9 pM of 153 or 153^{M} TAS3a transcripts. The error bars indicate SD (n=4). Asterisks denote a significant difference (t test, P<0.001). (G) Urea-polyacrylamide gel showing turnover and transport of 555 nt TAS3a transcript. The Col-0 leaves were infiltrated with 22.9 pM of 555 nt ³²p-TAS3a transcript and the RNA extracted from local and distal leaves was analyzed using RNA gel electrophoresis. (H) Real-time quantitative RT-PCR showing relative expression levels of TAS3a transcript in plants infiltrated with water or T-555 (0.3 nmol/ml RNA). The cDNA was amplified using primers that map to the 5′ (153 nt region) or 3′ regions of TAS3a (see FIG. 9A). The error bars indicate SD (n=4). Results are representative of two independent experiments. Asterisks denote a significant difference between water and T-555 treated leaves (t test, P<0.001). (I-J) Real-time quantitative RT-PCR showing relative expression levels of TAS3a in (I) local leaves and (J) PEX collected from mock (MgCl₂)- and avrRpt2-inoculated Col-0 and gly1 gli1 plants. The error bars indicate SD (n=4). Results are representative of three independent experiments. Asterisks denote a significant difference between mock- and avrRpt2 inoculated RNA (t test, P<0.001). (K) Real-time quantitative stem-loop RT-PCR showing relative expression levels of Tasi-ARFs D7 and D8 in Col-0 and gly1 gli1 plants treated with avrRpt2. The error bars indicate SD (n=3). Results are representative of two independent experiments. Asterisks denote a significant difference between 0 and 3 h post treatment (t test, P<0.005). (L) SAR response in distal leaves of Col-0, sid2, and gly1 gli1 plants treated locally with MgCl₂, avrRpt2, or 153 or 555 nt TAS3a transcripts. The virulent pathogen (DC3000) was inoculated 48 h post-local treatments. CFU indicates colony forming units. Asterisks denote a significant difference with respective mock-inoculated samples (t test, P<0.0001). (M-N) SAR response in distal leaves of Col-0 and (M) gly1 gli1 or (N) sid2 plants treated locally with MgCl₂ or Tasi-ARFs D7 or D8. The virulent pathogen (DC3000) was inoculated 48 h post-local treatments. Asterisks denote a significant difference with mock (t test, P<0.0001). The experiment was repeated three times with similar results. (O-Q) SAR response in Col-0 plants infiltrated with MgCl₂ (PEX_{MgCl2}) or avrRpt2 (PEX_{avrRpt2}) petiole exudates (PEX) collected from (O) Col-0 and gly1 gli1, (P) Col-0 and ago7, or (Q) Col-0 and tas3a plants. The distal leaves were inoculated with virulent pathogen at 48 h post infiltration of primary leaves. Asterisks denote a significant difference with mock (t test, P<0.0001). These experiments were repeated three times with similar results. (R) SAR response in Col-0 and arf plants infiltrated with MgCl₂ or avrRpt2.The distal leaves were inoculated with virulent pathogen at 48 h post infiltration of primary leaves. Asterisks denote a significant difference with mock (t test, P<0.0001). The experiment was repeated two times with similar results. (S) SAR response in Col-0 and transgenic plants expressing ARF3-GUS or a mutant form of ARF3-GUS (ARF3m-GUS) lacking the TAS3a cleavage site. The distal leaves were inoculated with virulent pathogen at 48 h post infiltration of primary leaves. Asterisks denote a significant difference with mock (t test, P<0.0001). The experiment was repeated three times with similar results. (T) SAR response in Col-0, ARF3-GUS and ARF3m-GUS after localized inoculation with avrRpt2 or TAS3a 153 or 555 nt transcripts. The distal leaves were inoculated with virulent pathogen at 48 h post infiltration of primary leaves. Asterisks denote a significant difference with mock (t test, P<0.0001). The experiment was repeated three times with similar results. (U) SAR response in Col-O plants infiltrated with MgCl₂ (PEX_{MgCl2}) or avrRpt2 (PEX_{avrRpt2}) petiole exudates (PEX) collected from Col-0 or ARF3m-GUS plants. The distal leaves were inoculated with virulent pathogen at 48 h post infiltration of primary leaves. Asterisks denote a significant difference with mock (t test, P<0.0001). These experiments were repeated three times with similar results.

FIGS. 5A-B show images illustrating Tas3a-mediated systemic signaling in plants. (A) Model for the biogenesis of Tasi-ARFs from TAS3a. Black line represents TAS3a transcript that contains 5′ ORF and mir390-AGO7 target sites. The biogenesis of Tasi-ARFs in initiated upon cleavage at 3′ miR390-AGO7 (A7) binding site (marked by an arrowhead). The resulting transcript is protected from degradation by SGS3 and subsequently transcribed by RDR6 into dsRNA. This dsRNA contains a 2-nt 3′-overhang and a 220-nt 5′-overhang. The 3′-overhang is optimal for DCL4 binding and results into processing of dsRNA from one end into phased siRNA duplexes with 2-nt 3′-overhangs. Ta-siRNAs are incorporated into RISC complex that contains AGO1 protein, which mediate target cleavage and inactivation. (B) A simplified model showing TAS3a-mediated systemic signaling in plants. Inoculation of avirulent pathogen triggers processing of TAS3a leading to generation of a 5′˜200-nt transcript and 21-nt Ta-siRNA. The 5′ transcript contains two overlapping ORFs that encode a 5-6 kd protein and is rapidly transported to the distal tissues. The Ta-siRNA target ARFs 2, 3 and 4, resulting in generation of an unknown factor designated as X. The tas3a, ago7, and gly1 gli1 plants are unable to generate X and remain defective in generation of the mobile signal. This suggests that transport of X is a prerequisite for SAR.

FIGS. 6A-I show graphs and images illustrating that overexpression of DRB2 compromises SAR without affecting SA or Pip levels. (A) Morphological phenotype of four-week-old Col-0 and transgenic plants overexpressing DRB1, DRB2, DRB3, DRB4, or DRB5. (B) Real-time quantitative RT-PCR showing relative expression levels of DRB genes in Col-0 and respective 35S-DRB plants. The error bars indicate SD (n=4). Results are representative of three independent experiments. Asterisks denote a significant difference between mock- and avrRpt2inoculated RNA (t test, P<0.001). (C-E) Protein immunoblots showing levels of (C) DRB1 or (D-E) DRB4 in Col-0 and 35S-DRB plants. Ponceau-S staining of the immunoblot was used as the loading control. The experiment was repeated three times with similar results. (F) Electrolyte leakage in Col-0 and 35S-DRB2 plants infiltrated with MgCl₂ or avrRpt2 Pst. Error bars represent SD (n=6). This experiment was repeated two times with similar results. (G) RNA gel-blot analysis showing relative expression levels of PR-1 in Col-0 and 35S-DRB2 plants after mock (MgCl₂)- and pathogen (avrRpt2)-inoculations. The leaves were sampled 48 h post treatments and the experiment was repeated two times with similar results. (H) SA levels in local tissues of Col-0 and 35S-DRB2 plants after mock (MgCl₂)- and pathogen (avrRpt2)-inoculations. The leaves were sampled 48 h post treatments and the experiment was repeated two times with similar results. Asterisks denote a significant difference with respective mock-inoculated samples (t test, P<0.0001). (I) Pip levels in local tissues of Col-0 and 35S-DRB2 plants after mock- and avrRpt2-inoculations. The leaves were sampled 48 h post treatments. Asterisks denote a significant difference with mock (t test, P<0.0001). The experiment was repeated three times with similar results.

FIGS. 7A-B show graphs illustrating that components of RNA silencing pathway are required for SAR. (A) Local resistance of ago1 and ago7 plants to virulent Pst DC3000 and avirulent avrRpt2 pathogens. Plants lacking the R protein RPS2 were used as a control. Leaves were sampled 3 days post inoculation. Asterisks denote a significant difference (t test, P<0.0001). The experiment was repeated three times with similar results. (B) SAR response in distal leaves of Col-0, dcl4, sgs3, and rdr6 plants treated locally with MgCl₂, or avrRpt2. The virulent pathogen (DC3000) was inoculated 48 h post-local treatments. CFU indicates colony forming units. Asterisks denote a significant difference with respective mock-inoculated samples (t test, P<0.0001).

FIGS. 8A-E show graphs and images illustrating that tas3a plants show normal local resistance, HR, and PR-1 expression. (A) Real-time quantitative RT-PCR showing relative expression levels of TAS3a in Col-0 and respective tas mutants. The error bars indicate SD (n=4). Results are representative of two independent experiments. Asterisks denote a significant difference (t test, P<0.001). (B) Typical morphological phenotypes of DC3000 inoculated distal leaves of Col-0 and tas3a plants. The local leaves of these plants were inoculated with mock (MgCl₂) and avrRpt2 48 h prior to DC3000 inoculation. (C) Local resistance of Col-0 and tas3a plants to avirulent avrRpt2 pathogen. Error bars represent SD (n=4). The experiment was repeated three times with similar results. (D) Electrolyte leakage in Col-0 and tas3a plants infiltrated with MgCl₂ or avrRpt2 Pst. Error bars represent SD (n=6). This experiment was repeated two times with similar results. (E) Real-time quantitative RT-PCR showing relative expression levels of PR-1 in mock- and avrRpt2-inoculated Col-0 and tas3a leaves. The error bars indicate SD (n=4). Results are representative of two independent experiments. Asterisks denote a significant difference between mock and avrRpt2 inoculated RNA (t test, P<0.001).

FIGS. 9A-H show images and graphs illustrating that TAS3a RNA contains two overlapping open reading frames. (A) Nucleotide sequence of mature TAS3a RNA (SEQ ID NO: 1). Region shaded indicates overlapping open reading frames. The last two segments of shaded Nucleotides (23 nt and 20 nt segments) are miR390-AGO7 targeting sites on TAS3a transcript. (B) Real-time quantitative RT-PCR showing relative expression levels of ARF3 in Col-0 plants treated with buffer or Tas3a₅₅₅ for 24 h. The error bars indicate SD (n=4). Results are representative of two independent experiments. Asterisks denote a significant difference (t test, P<0.0001). (C) Uptake assays showing percentage of ³²P-TAS3a₅₅₅ transported into isolated protoplasts. Fresh protoplasts (10⁶/ml) were incubated with 2 μM ³²P-TAS3a₅₅₅ for 1 h, analyzed microscopically before and after four washes and quantified for the amount of radiolabel. The experiment was repeated three times with similar results. (D) RNA gel electrophoresis of in vitro synthesized TAS3a transcripts used in this study. (E) Typical morphological phenotypes of DC3000 inoculated distal leaves of Col-0 and tas3a plants. The local leaves of these plants were inoculated with mock (MgCl₂), avrRpt2, or TAS3a 555 bp transcript 48 h prior to DC3000 inoculation. (F) SAR response in distal leaves of Col-0 plants treated locally with MgCl₂, avrRpt2 or indicated concentrations of TAS3a transcripts. The virulent pathogen (DC3000) was inoculated 48 h post-local treatments. CFU indicates colony forming units. Asterisks denote a significant difference with respective mock-inoculated samples (t test, P<0.0001). (G) Amino acid sequence of the putative protein encoded by the ORFs contained in TAS3a (SEQ ID NO: 11). Shaded amino acids indicate translational start sites of the two overlapping ORFs. (H) Thermodynamic ensemble predictions of the wild-type (AUG start codon) and mutant (AUU start codon) 153 bp RNA (SEQ ID NO: 8) showing minimum free energy secondary structures. The structural predications were carried out using RNAfold webserver.

FIGS. 10A-B show an image and a graph illustrating that Tasi-ARFs D7 and D8confer SAR. (A) Typical morphological phenotypes of DC3000 inoculated distal leaves of Col-0 plants. The local leaves of these plants were inoculated with mock (MgCl₂), TAS3a₅₅₅ or Tasi-ARFs D7 or D8 prior to DC3000 inoculation. (B) Real-time quantitative stem-loop RT-PCR showing relative expression levels of Tasi-ARF D7 in Col-0, ago7 and 35S-DRB2 plants. The error bars indicate SD (n=3). Results are representative of two independent experiments. Asterisks denote a significant difference (t test, P<0.005).

FIGS. 11A-E show graphs illustrating that RNAase treatment of PEX degrades TAS3a but does not affect SA or AzA levels. (A) Real-time quantitative RT-PCR showing relative expression levels of TAS3a in mock- and avrRpt2-inoculated Col-0 leaves 24 h post inoculation. The error bars indicate SD (n=4). Results are representative of four independent experiments. Asterisks denote a significant difference (t test, P<0.0001). (B) Real-time quantitative RT-PCR showing relative expression levels of TAS3a in PEX_{MgCl2}, PEX_{avrRpt2} and RNAase treated PEX_{avrRpt2}. The error bars indicate SD (n=4). Results are representative of two independent experiments. Asterisks denote a significant difference (t test, P<0.0001). (C) SA and (D) AzA levels in PEX_{MgCl2}, PEX_{avrRpt2} and RNAase treated PEX_{avrRpt2}. The error bars indicate SD (n=4). Results are representative of two independent experiments. Asterisks denote a significant difference from PEX_{MgCl2} (t test, P<0.001). (E) Percentage of ³²p-TAS3a associated radiolabel detected in the distal tissues of Col-0 plants infiltrated with 153 and 555 bp transcripts. Asterisks denote a significant difference (t test, P<0.001). The error bars indicate SD (n=4).

FIGS. 12A-D show graphs and images illustrating that exogenous application of TAS3a induces G3P but not SA, Pip, or ROS. (A) SA (left panel) and SAG (right panel) levels in Col-0 plants infiltrated with MgCl₂, avrRpt2 or TAS3a transcripts. Leaf samples were collected 24 and 48 h post treatments. The error bars indicate SD (n=4). Asterisks denote a significant difference with respective mock-inoculated samples (t test, P<0.0001). (B) Pip levels in local tissues of Col-0 plants infiltrated with MgCl₂ (mock), avrRpt2 or TAS3a transcripts. The leaves were sampled 48 h post treatments. Asterisks denote a significant difference with mock (t test, P<0.0001). The experiment was repeated two times with similar results. (C) H₂O₂ levels in local tissues of Col-0 plants after mock (MgCl₂), pathogen (avrRpt2) or TAS3a treatments. The leaves were sampled 24 h post treatments and stained with DAB (3, 3-diaminobenzidine). The experiment was repeated two times with similar results. (D) G3P levels in local tissues of water or 555 bp TAS3a transcript treated Col-0 plants. The leaves were sampled 24 h post treatments and the experiment was repeated two times with similar results. Asterisks denote a significant difference with respective mock-inoculated samples (t test, P<0.001).

FIGS. 13A-B show images illustrating that the gly1 gli1 plants show zippy phenotype. (A) Morphological phenotypes of three-week-old Col-0, gly1 gli1 and tas3a plants. (B) Morphological phenotype of four-week-old Col-0 and gly1 gli1 plants showing early flowering phenotype of gly1 gli1 plants.

FIGS. 14A-C show a graph and images illustrating that increased expression of ARF3 leads to zippy phenotype. (A) Real-time quantitative RT-PCR showing relative expression levels of ARF3 in Col-0 and transgenic plants expressing wild-type or mutant forms of ARF3-GUS under the ARF3 promoter. The error bars indicate SD (n=4). Results are representative of two independent experiments. Asterisks denote a significant difference from ARF3 levels in Col-0 (t test, P<0.001). (B) GUS Histochemical staining showing relative levels of GUS in ARF3-GUS and ARF3m-GUS plants. The leaves were stained with X-gluc (5-Bromo-4-chloro-3-indoxyl-beta-D-glucuronide) and Col-0 leaves were used as a negative control. (C) Typical morphology phenotype shown by 3- and 4-week-old Col-0, ARF3-GUS and ARF3m-GUS plants.

FIGS. 15A-D show graphs and images illustrating that increased expression of ARF3 is not associated with increased SA, Pip, ROS, or G3P levels. (A) SA and (B) Pip levels in Col-0, ARF3-GUS, and ARF3m-GUS plants infiltrated with MgCl₂ or avrRpt2. Leaf samples were collected 24 and 48 h post treatments. The error bars indicate SD (n=4). Asterisks denote a significant difference with respective mock-inoculated samples (t test, P<0.0001). (C) H₂O₂ levels in local tissues of Col-0, ARF3-GUS and ARF3m-GUS plants after mock (MgCl₂) and pathogen (avrRpt2) inoculations. The leaves were sampled 24 h post treatments and stained with DAB (3, 3-diaminobenzidine). The experiment was repeated two times with similar results. (D) G3P levels in local tissues of Col-0 and ARF3m-GUS plants after mock (10 mM MgCl₂)- and pathogen (avrRpt2)-inoculations. The leaves were sampled 24 h post treatments and the experiment was repeated two times with similar results. Asterisks denote a significant difference with respective mock-inoculated samples (t test, P<0.001).

FIG. 16 shows a graph illustrating that the tas3a plants show increased expression of ARF2, 3, and 4. Real-time quantitative RT-PCR showing relative expression levels of ARF genes in Col-0 and tas3a plants. The error bars indicate SD (n=4). Results are representative of two independent experiments. Asterisks denote a significant difference Col-0 and tas3a plants (t test, P<0.0001).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DEFINITIONS

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All combinations of method or process steps as used herein can be performed in any order, unless otherwise specified or clearly implied to the contrary by the context in which the referenced combination is made.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, ElZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

As used herein, the term “patient” refers to a subject afflicted with a disease or disorder. A patient includes human and veterinary subjects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. It is also contemplated that, in certain aspects, unless expressly indicated to the contrary, individual substituents can be further optionally substituted (i.e., further substituted or unsubstituted).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Provided herein are compounds for conferring systemic acquired resistance (SAR) in plants. In some embodiments, the compound includes a nucleotide sequence relating to or derived from trans-acting small interfering RNA3a (TAS3a). In some embodiments, for example, the compound includes the TAS3a gene having the sequence according to SEQ ID NO: 1. In some embodiments, the compound includes the RNA transcript of TAS3a having the sequence according to SEQ ID NO: 2. In some embodiments, the compound includes a portion of the gene or RNA transcript of TAS3a. For example, in some embodiments, the compound includes a Ta-siRNA that negatively regulates auxin response factors (Tasi-ARF), such as, but not limited to, the 21 nucleotide (21-nt) Tasi-ARF according to SEQ ID NO: 3 and/or SEQ ID NO: 4. In some embodiments, the compound includes an open reading frame (ORF), such as, but not limited to, the ORF according to SEQ ID NO: 5, SEQ ID NO: 6, SEQ ID NO: 7, and/or SEQ ID NO: 8. In some embodiments, the compound includes a truncated portion of TAS3a with the 5′ miR390-AGO7 target site, such as, but not limited to, the truncated 3′ portion according to SEQ ID NO: 9 or SEQ ID NO: 10.

In some embodiments, the compound includes one or more of the sequences disclosed herein having at least one nucleotide mutation. The at least one nucleotide mutation may include a single nucleotide substitution or deletion, two nucleotide substitutions or deletions, three nucleotide substitutions or deletions, or more than three nucleotide substitutions or deletions. As will be appreciated by those skilled in the art, depending upon the location, any such number of mutations may be included in the sequence without negatively impacting the SAR conferring ability of the compound.

Additionally or alternatively, in some embodiments, the compound includes one or more of the sequences disclosed herein having at least one modification. The at least one modification may include a ribose 2′/3′ modification in an RNA sequence, a 3′-end modification in an RNA sequence, a locked nucleic acids (LNA) modification in an RNA sequence, and/or conjugation of a nanoparticle (NP) to the sequence. In one embodiment, the 2′-ribose modification includes 2′-fluorination, 2′-oxymethilation, 2′amination of pyrimidines, any other suitable 2′-ribose modification, or a combination thereof. In another embodiment, the 2′/3′-ribose modification increases RNA stability (e.g., protects RNA from nuclease degradation) without sacrificing potency. In one embodiment, the 3′-end modification includes replacing the 3′-end phosphate group with phosphothioate or boranophosphate. In one embodiment, LNA includes forming methyl linkages between the ribose's 2′- and 4′-positions in an RNA sequence. In another embodiment, LNA modification increases RNA nuclease resistance without affecting compatibility with the RNAi machinery, increases hybridization affinity with mRNA, and/or decreases off-target effects. In one embodiment, the NP conjugation includes any suitable conjugation according to known methods of NP based delivery of RNA, such as, but not limited to, the methods used in treatment of cancers in humans. In another embodiment, the NP conjugation improves stability of the RNA and/or presents specific physical and chemical properties that assist nucleic acids in entering cells.

Also provided herein, in some embodiments, are methods of conferring SAR. In some embodiments, the method includes administering one or more of the compounds disclosed herein. In some embodiments, for example, the method includes exogenous application of a compound having a nucleotide sequence relating to or derived from trans-acting small interfering RNA3a (TAS3a). In one embodiment, the compound includes an isolated sequence according to one or more of the sequences disclosed herein. Alternatively, in one embodiment, the compound includes one or more of the sequences disclosed herein having at least one mutation or modification thereto. In some embodiments, the exogenous application of these compounds induces robust SAR in transgenic, mutated, modified, and/or wild-type plants. In some embodiments, the exogenously applied TAS3a is a SAR-associated signal that functions downstream of all known signals. Without wishing to be bound by theory, it is believed that TAS3a induces SAR by downregulating auxin response factors (ARFs) 2, 3, and 4, whereas increased expression of ARF3 compromises SAR in a TAS3a-independent manner. Additionally or alternatively, in some embodiments, glycerol-3-phosphate (G3P) is present and/or administered for TAS3a stability.

In some embodiments, the RNA undergoes truncation following administration. Without wishing to be bound by theory, it is believed that the truncated RNA is the only species that moves from local to distal tissues. In some embodiments, the truncated RNA includes an ORF (SEQ ID NO: 8) which encodes a protein (SEQ ID NO: 11) that facilitates generation of small RNA. Alternatively, in some embodiments, SAR may be induced by localized application of the small RNA. Following administration, exogenous RNA does not induce non-specific defense responses and therefore does not lead to any developmental phenotypes. Additionally, the RNA and/or downstream factors regulated by the RNA can be used at a commercial scale to elicit broad-spectrum immunity against plant pathogens and pests. Accordingly, in some embodiments, the method includes administering one or more of the compounds disclosed herein to field grown plants to confer enhanced disease resistance, enhanced resistance against microbial pathogens, resistance to soil-born pathogens and pests, and/or SAR in plants without affecting yield. In some embodiments, the method replaces chemical based control of plant diseases.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

This Example focuses on the discovery that trans-acting small interfering RNA3a derived sRNA regulates systemic acquired resistance in Arabidopsis. Systemic acquired resistance (SAR) is a type of broad-spectrum resistance that involves the generation of an as yet unidentified signal at the primary infection site, which transport systemically to arm distal parts against subsequent infections. This Example shows that trans-acting small interfering RNA3a (TAS3a) is the previously unidentified SAR-associated signal that functions downstream of all known signals and is required for the generation of the mobile signal.

In particular, this Example shows that the TAS3a mature transcript is processed to generate 21-nt Ta-siRNA and a 3′ truncated transcript, which is rapidly transported to distal tissues. The TAS3a transcript and thereby Ta-SiRNA levels are regulated by the SAR inducer glycerol-3-phosphate. Ta-siRNA negatively regulate auxin response factors (ARF) and consequently, plants overexpressing ARF3 show compromised SAR. Knock-out mutation in TAS3a or RNA silencing components contributing to Tasi-ARF biosynthesis also compromises SAR, but without altering levels of chemical signals generally associated with SAR. Conversely, exogenous application of mature TAS3a transcript, its 5′ protein encoding region, the 3′ region containing the microRNA390-Argonaute7 targeting sites, or the Tasi-ARFs induces robust SAR. Together, the results described herein show that the developmental signal TAS3a functions as an important regulator of SAR.

### Discussion

Systemic acquired resistance (SAR) is a form of systemic immunity that protects distal uninfected parts of the plant against secondary infections. SAR involves the generation of mobile signals in the primary infected leaves, which when translocated to distal uninfected portions, activate defense responses resulting in disease resistance. A number of chemical SAR inducers have been identified including salicylic acid (SA), pipecolic acid (non-protein amino acid derivative of lysine, Pip), azelaic acid (C₉ dicarboxylic acid, AzA), glycerol-3-phosphate (phosphorylated sugar alcohol derivative, G3P), nitric oxide (NO), and reactive oxygen species (ROS). Recent analysis has shown that Pip functions upstream of the AzA-G3P branch to confer SAR by inducing the biosynthesis of free radicals. AzA functions upstream of G3P and the Pip-NO-ROS-AZA-G3P branch functions in parallel to SA-derived signaling during SAR. Transport of SA from primary infected tissue to the distal tissue occurs via the apoplast (space between cell wall and plasma membrane). In contrast, G3P and AzA are transported preferentially via plasmodesmata (PD). Transport of both SA and G3P is essential for Pip accumulation in the distal tissue and for SAR. This suggests that coordinated transport and feed-back regulation amongst various chemical signals is an important aspect of SAR activation.

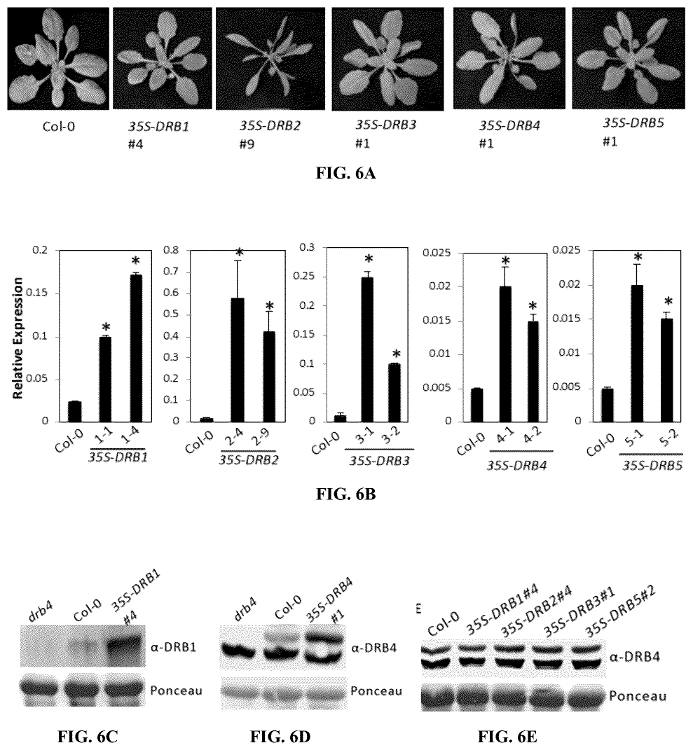

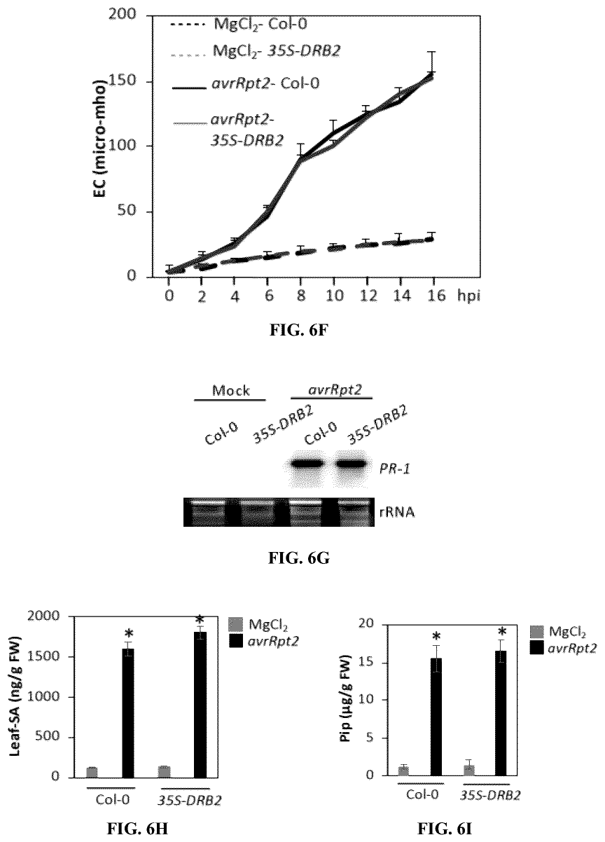

SAR also requires a number of proteins, including double-stranded RNA binding (DRB) proteins 1, 2, and 4. In view of this, together with the previously demonstrated antagonistic relationship between DRB2 and DRB4 (characterized based on levels of polymerase IV dependent siRNA), the present inventors assayed the effects of DRB overexpression on SAR. Transgenic Col-O plants expressing DRB proteins 1, 2, 3, 4, and 5 were generated via the 35S promoter and screened for respective transgene expression levels (FIGS. 6A-B). At least two independent transgenic lines per transgene were analyzed (FIG. 6B). Transgene overexpression corresponded to increased accumulation of DRB1 and DBR4 proteins in the respective transgenic lines (FIGS. 6C-D). Protein levels for DRB2, 3, and 5 could not be assayed because antibodies against these proteins showed non-specific cross reactivity to multiple bands on Western blots. Nevertheless, presence of the zippy phenotype (characterized by narrow leaves) in the 35S-DRB2 plants confirmed DRB2 overexpression as previously reported (FIG. 6A).

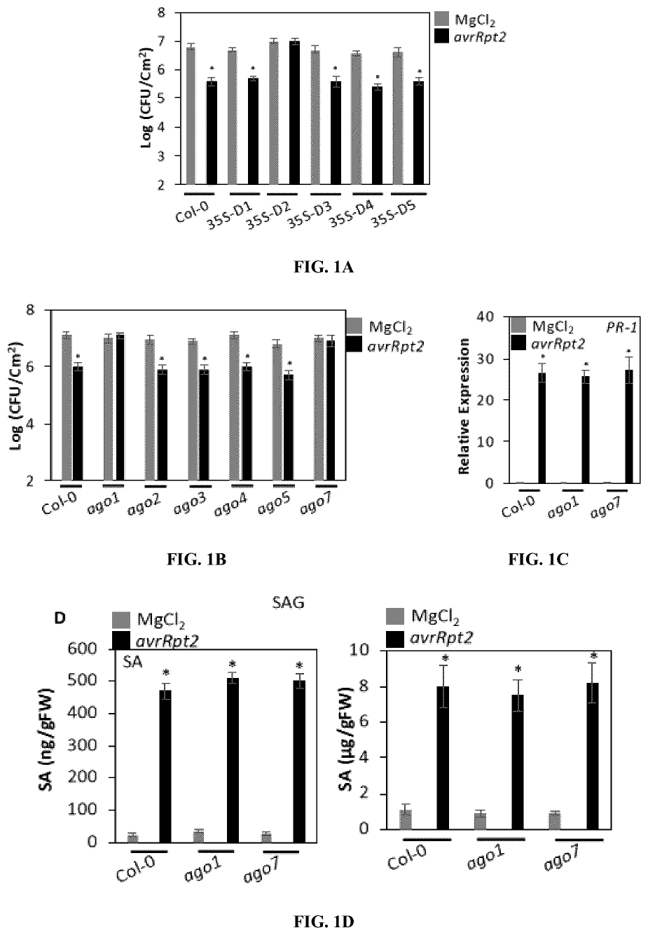

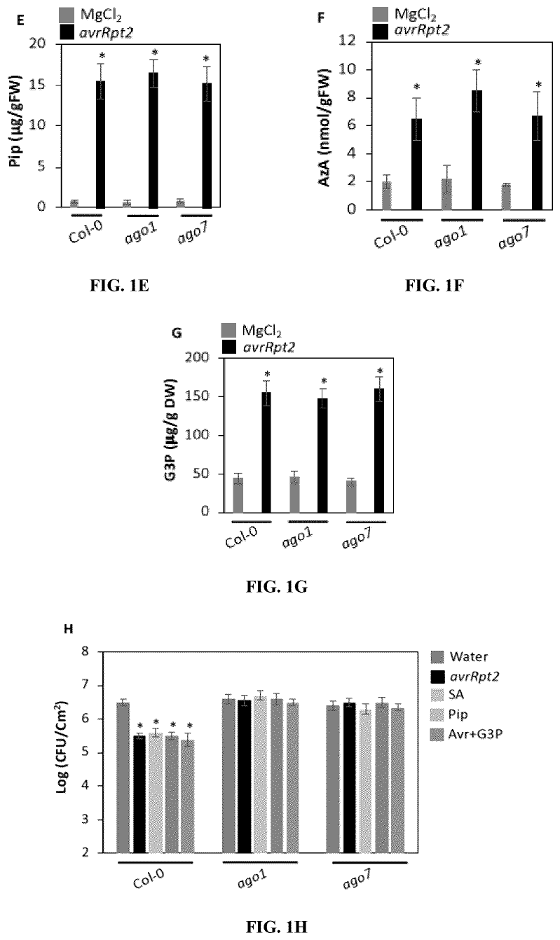

Notably, the zippy phenotype of 35S-DRB2 plants was similar to the morphological phenotype of the drb4 mutant, suggesting that increased DRB2 expression might impair DRB4 activity or DRB4-mediated signaling because 35S-DRB2 plants contained wild-type levels of DRB4 (FIG. 6E). The drb4 mutant is compromised in SAR, therefore the present inventors assayed SAR in 35S-DRB2 and other DRB overexpressing lines. Interestingly, only 35S-DRB2 plants were compromised in SAR (FIG. 1A), even though these plants showed normal HR and PR-1 induction (FIGS. 6F-G) as well as wild-type like levels of SA and Pip (FIGS. 6H-I) in their infected leaves. Together, these results suggested that a factor other than SA or Pip was responsible for the compromised SAR phenotype of 35S-DRB2 plants.

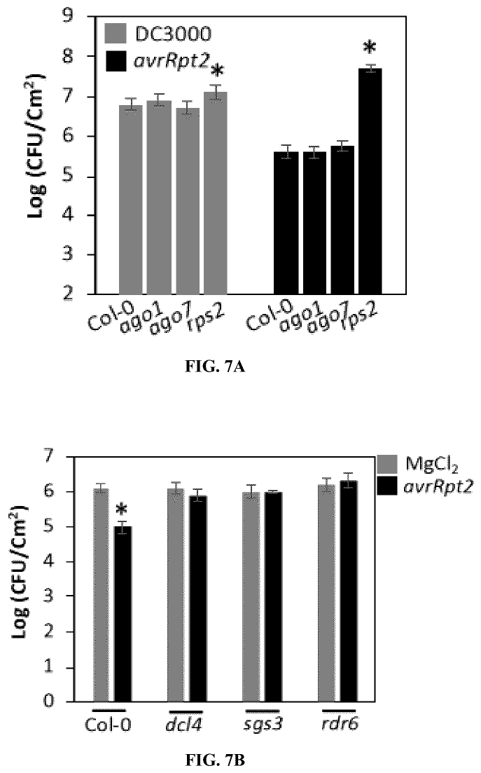

To test if the overexpression of DRB2 compromised SAR via its effect on a putative RNA signal, SAR was assayed in mutants defective in the RNA silencing pathway. For instance, SAR was tested in Argonaute [AGO, central regulators in the RNA silencing pathway] mutants. Of the six different ago mutants tested only ago1 and ago7 were compromised in SAR (FIG. 1B), although they showed wild-type like local resistance (FIG. 7A). Both ago1 and ago7 mutants also induced wild-type like levels of the SA marker PR-1 (FIG. 1C), and accumulated wild-type like levels of SA and its glucoside SAG (FIG. 1D). The ago1 and ago7 mutants also accumulated wild-type-like levels of Pip (FIG. 1E), AzA (FIG. 1F), or G3P (FIG. 1G). Consistent with these results, localized application of SA, Pip, or G3P were unable to restore SAR in ago1 or ago7 plants (FIG. 1H), suggesting that the importance of AGO1 and AGO7 proteins in SAR was not associated with SA-, Pip-, G3P-derived signaling. Besides ago1 and ago7, mutations in DCL4, SGS3 and RDR6, which generally operate upstream of AGO proteins, also compromised SAR (FIG. 7B). Together, these results suggested that RNA biogenesis and thereby possibly an RNA species was essential for SAR.

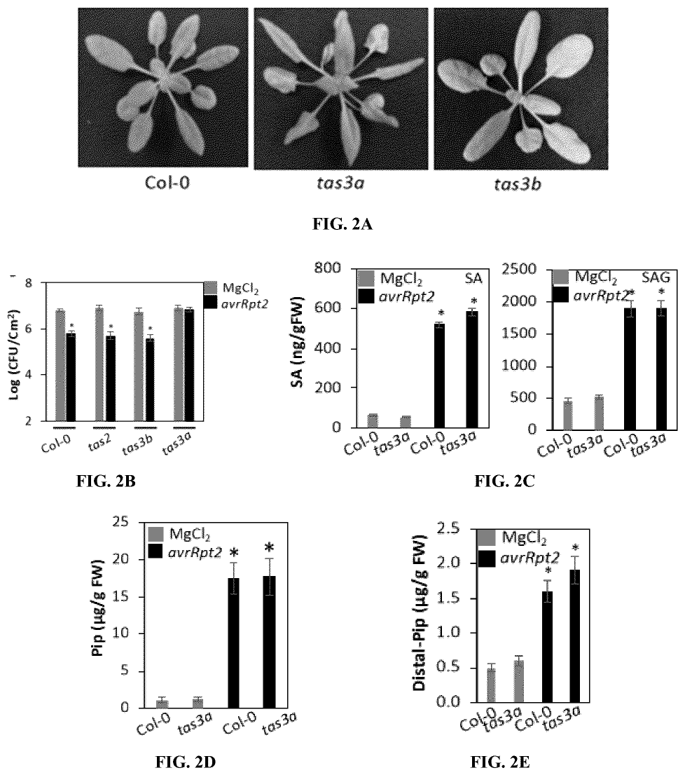

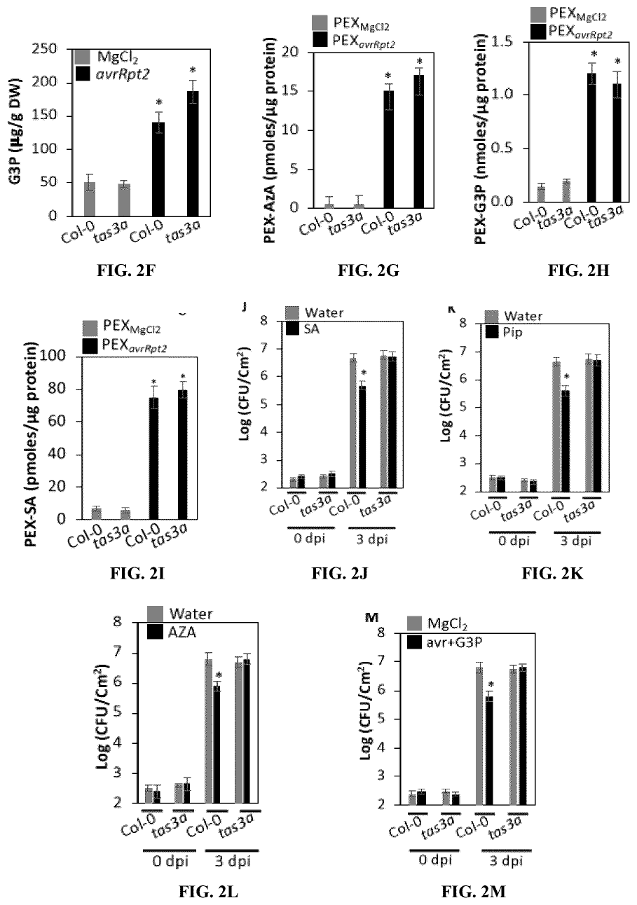

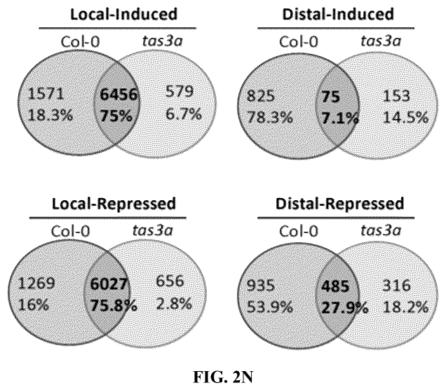

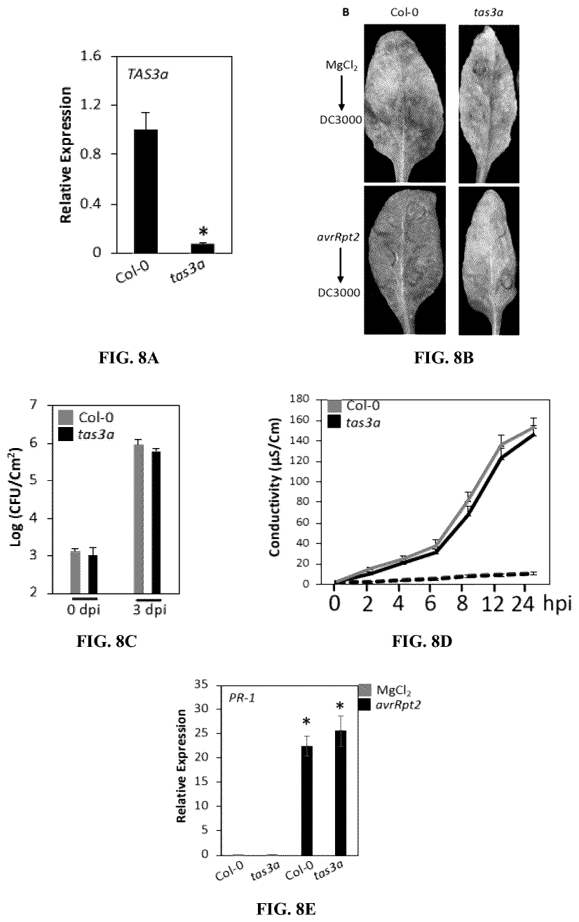

Overexpression of DRB2 has previously been shown to antagonize the DRB4-mediated synthesis of trans-acting RNA3a (TAS3a). Likewise, both AGO1, AGO7, DCL4, RDR6 and SGS3 are also involved in the biosynthesis of small(S) RNA generated from TAS3a. This raised the possibility that the compromised SAR phenotype of 35S-DRB2, ago1, and ago7 plants may be associated with reduced levels of TAS3a-derived sRNA. To assess this, SAR was first assayed in a previously characterized T-DNA knockout (KO) line of TAS3a. The tas2a plants were compromised in SAR, whereas KO mutations in TAS2 or TAS3b did not inhibit SAR (FIGS. 2A-B and 8A-B). Like 35S-DRB2 plants, the tas3a mutant showed zippy phenotype (FIG. 2A) and wild-type like local resistance and HR (FIGS. 8C-D), suggesting that TAS3a specifically contributed to SAR. The tas3a plants showed wild-type like PR-1 induction (FIG. 8E); accumulated normal levels of SA/SAG (FIG. 2C), local (FIG. 2D) and distal (FIG. 2E) Pip, and G3P (FIG. 2F); and showed normal transport of AzA, G3P, and SA (FIGS. 2G-I). Together these results suggested that the SAR defect of tas3a mutant was not associated with defects in the SA or Pip-AzA-G3P branches of the SAR pathway. Consistent with these results, and similar to the ago1 and ago7 mutants, localized application of SA, Pip, AzA, or G3P did not restore SAR in tas3a plants (FIGS. 2J-M). These results further suggested that TAS3a likely functioned downstream or independent of SA, Pip, AzA, and G3P.

The above results emphasized the importance of TAS3a in distal tissues. To assess this further genome-wide expression analysis of local and distal tissues from Col-0 and tas3a plants was carried out. Expression profiling showed ˜75% overlap in differentially expressed genes in the infected tissue, but only 7.1% and 27.9% overlap in induced and repressed genes in the distal tissue, respectively (FIG. 2N, Tables 1-4).

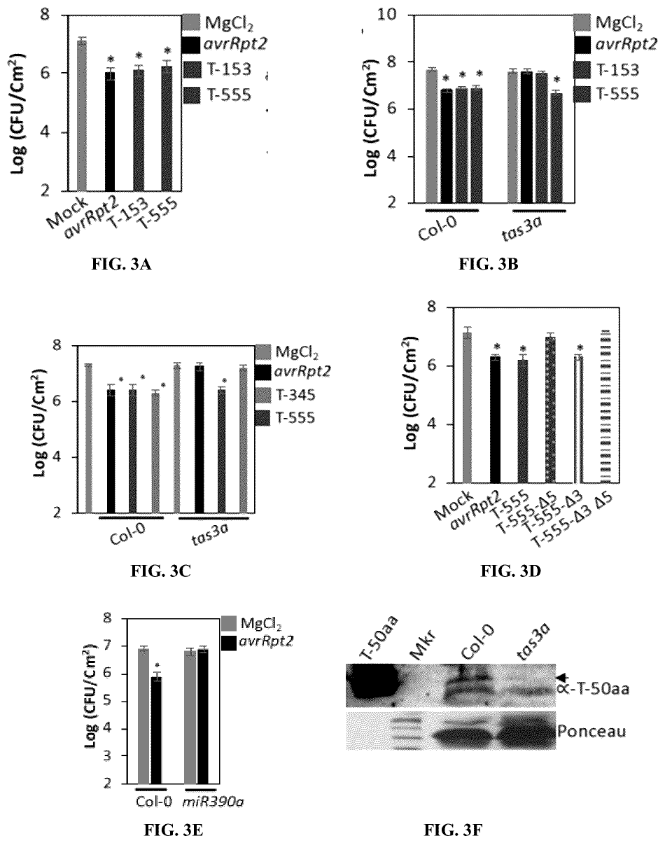

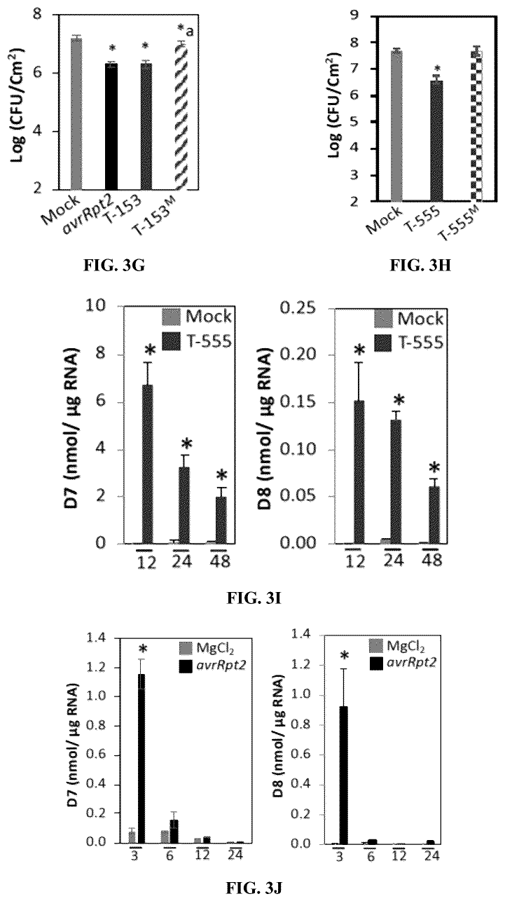

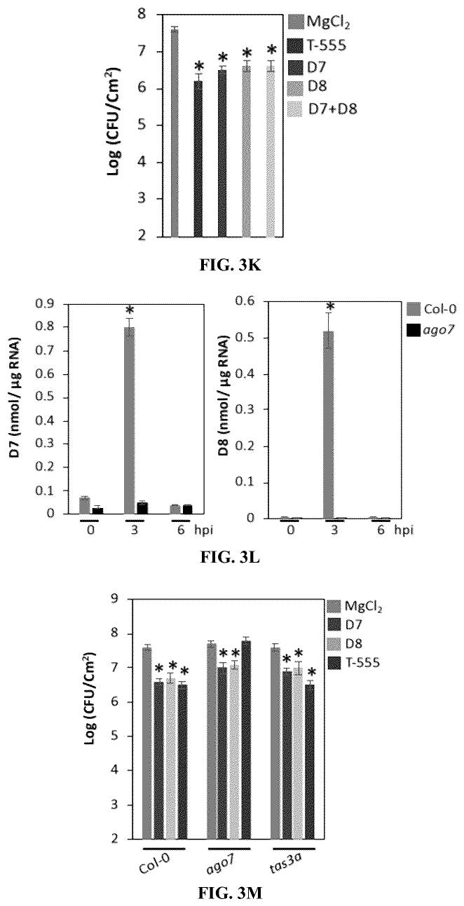

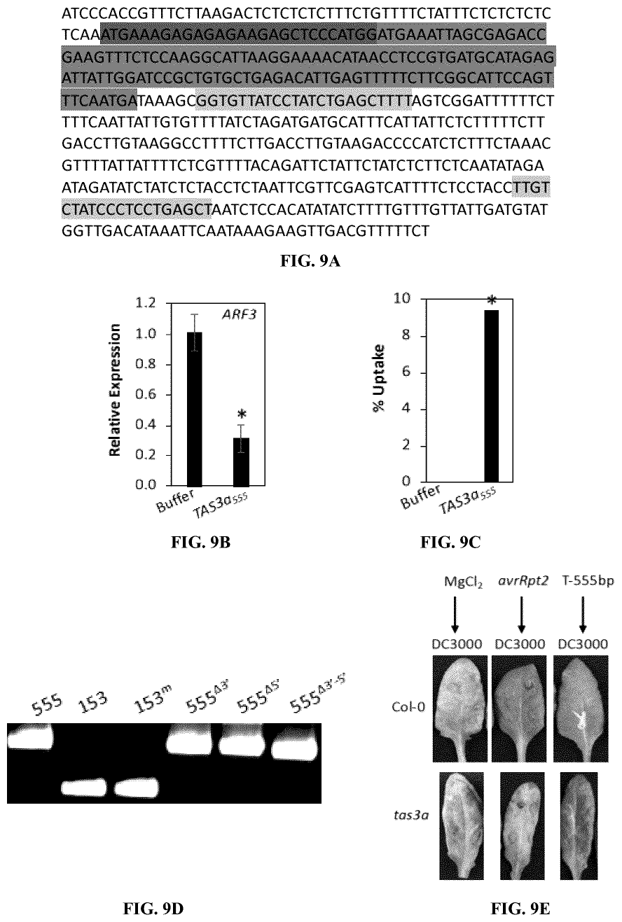

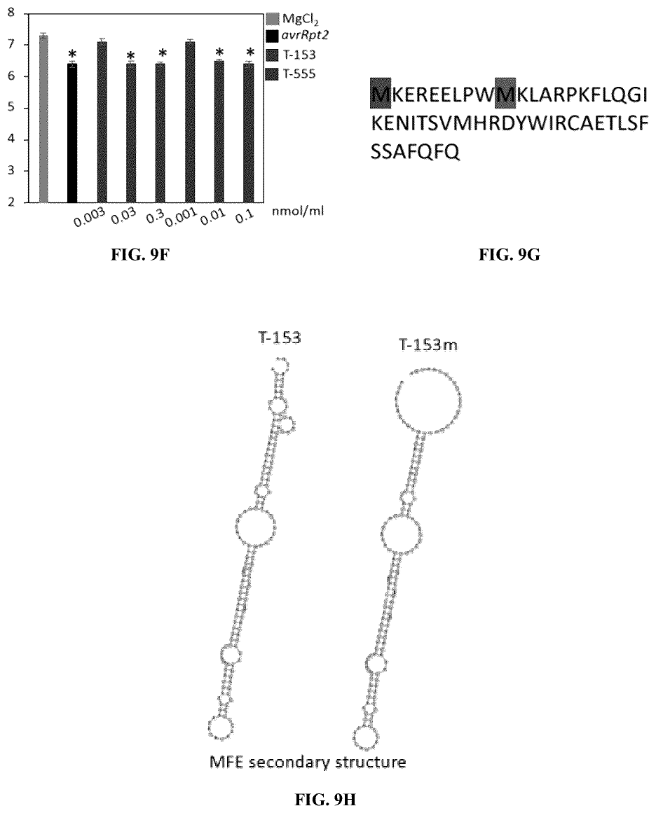

To test if TAS3a RNA itself served as the SAR inducer, in vitro transcribed TAS3a transcripts were tested in SAR assays. TAS3a encodes a 555 nucleotide (nt) mature transcript that contains two staggered open reading frames (ORF), 126 and 153 nt in length each (FIG. 9A). Exogenous application of TAS3a transcript suppressed expression of TAS3a target gene auxin responsive factor (ARF) 3 (FIG. 9B), suggesting that infiltrated RNA was able to enter into the cells. This was further confirmed by uptake of TAS3a transcript in isolated protoplasts (FIG. 9C). The 153 and 555 nt transcripts were assayed for SAR and interestingly, when applied locally, both were able to induce robust SAR in wild-type plants (FIGS. 3A and 9D-E) and only ˜0.3 nmol/ml RNA was sufficient to induce SAR (FIG. 9F). More importantly, the 555 nt (Tas3a₅₅₅), but not 153 nt TAS3a transcript (Tas3a₁₅₃) was able to reconstitute SAR on tas3a mutant plants (FIG. 3B). These results indicate that Tas3a₁₅₃-induced SAR in wild-type plants is dependent on the presence of the Tas3a₅₅₅ transcript, which is absent in the tas3a mutant.

The 555 nt transcript contains two miR390/AGO7 targeting sites downstream of the 153 nt ORF (FIG. 9A), that are involved in small RNA biogenesis. Thus, it was possible that the 153 nt ORF functioned in trans with the remainder 345 nt 3′ end of TAS3a, to confer SAR. To test this, SAR was assayed in Col-0 and tas3 plants treated with only the 345 nt 3′ end of TAS3a transcript (Tas3a₃₄₅). Like Tas3a₁₅₃, localized infiltration of Tas3a₃₄₅ also induced SAR in Col-0, but not tas3a plants (FIG. 3C). Together, these results suggested that: a) presence of the full length Tas3a Tas3a₅₅₅ transcript was a prerequisite for either Tas3a₁₅₃ or Tas3a₃₄₅ induced SAR and; b) increasing the amount of either the Tas3a₁₅₃ or the Tas3a₃₄₅ transcripts induced SAR in the wild-type background. We tested the importance of the miR390/AGO7 targeting sites by assaying the SAR inducing ability of Tas3a₅₅₅ transcript lacking one or both miR390-AGO7 targeting sites (FIG. 9A). Mutant Tas3a₅₅₅ transcripts with 5′ (Tas3a_{555-Δ5}) or both 3′ and 5′ targeting sites deleted (Tas3a_{555-Δ3 Δ5}) were unable to induce SAR on wild-type plants (FIG. 3D). In comparison, mutant Tas3a₅₅₅ transcript lacking the 3′ cleavage site induced normal SAR (FIG. 3D). Together, these results suggest that TAS3a 5′ miR390-AGO7 target site is more crucial for SAR. This was further consistent with the fact that mutations in miR390a, which binds the TAS3a 5′ and 3′ target sites, also compromised SAR (FIG. 3E). Together, these results supported the notion that processing of the Tas3a₅₅₅ transcript at the 5′ miR390-AGO7 target site was important for the SAR-inducing ability of Tas3a. Furthermore, besides the full-length Tas3a₅₅₅ transcript, the Tas3a₁₅₃ transcript is also an important accessory for SAR.

The 153 nt ORFs present in the Tas3a₅₅₅ transcript have been proposed to encode a protein that promotes sRNA biogenesis from TAS3a. This suggested that the peptide encoded by the 153 nt ORF could be important for TAS3a-mediated SAR. Although leaderless 153 nt ORF were used for the SAR assays, such transcripts are translatable in eukaryotic systems. A second possibility is that the translatable product of the 126 nt ORF within the 153 nt transcript is essential for SAR (FIG. 9A). These possibilities were tested by first generating polyclonal antibodies against a 50 amino acid peptide derived from the 153 nt ORF (FIG. 9G). Notably, these antibodies detected an ˜5-6 kD band in protein extracts from wild-type plants, levels of which were significantly reduced in the tas3a mutant (FIG. 3F). The importance of this peptide was tested by generating an untranslatable mutant form of Tas3a₁₅₃ (containing a G to U change in the AUG start codon) and using it in the SAR assays. Interestingly, Tas3a_{153M} showed drastically reduced SAR-inducing ability (FIG. 3G). Likewise, Tas3a₅₅₅ mutated in AUG was unable to confer SAR on wild-type plants (FIG. 3H). Although these results support the notion that translation of the Tas3a₁₅₃ transcript is important for SAR, it should be noted that the single base change from AUG to AUU does alter the secondary structure (minimum free energy structure) of the Tas3a₁₅₃ RNA (FIG. 9H). Thus, it is also possible that the altered secondary structure of the Tas3a_{153M} RNA, rather than its translatability, renders it less effective inducer of SAR.

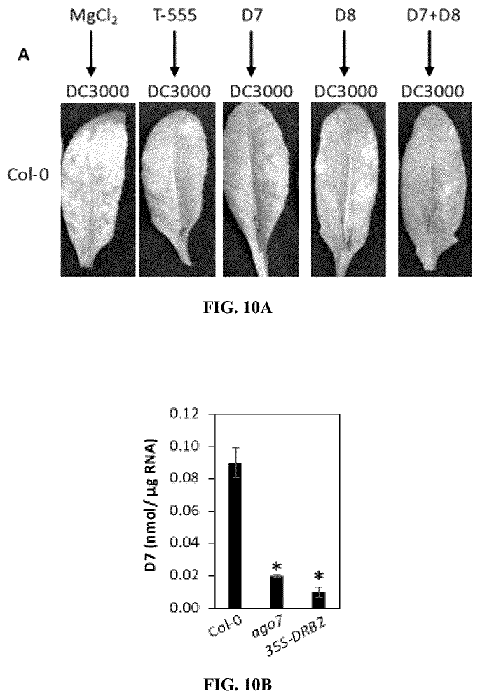

It was possible that exogenous application of Tas3a₅₅₅ transcript conferred SAR by increasing 21 nt sRNA designated as Tasi-ARFs. To test this, Tasi-ARFs levels were first assayed in plants treated with Tas3a₅₅₅ transcript. A time-course analysis of two Tasi-ARFs, designated D7 and D8, showed that these were induced within 12 h of treatment and their levels gradually declined at later time points (FIG. 3I). Another time-course analysis showed that D7 and D8 were induced within 3 h post inoculation of Pst avrRpt2 on wild-type plants (FIG. 3J). Furthermore, exogenous application of either D7 or D8 conferred robust SAR on wild-type plants (FIGS. 3K and 10A). Together, these results suggested that exogenous TAS3a likely conferred SAR by increasing the Tasi-ARF levels, and this in turn was consistent with reduced Tasi-ARF levels in 35S-DRB2 and ago7 plants (FIG. 10B). Consistent with their proposed function in generation of Tasi-ARF, Pst avrRpt2 inoculation was unable to induce D7 or D8 levels on ago7 plants (FIG. 3L). Moreover, exogenous D7 or D8, but not Tas3a₅₅₅ transcript, were able to restore SAR in ago7 plants (FIG. 3M). The D7 and D8 also conferred SAR on tas3a plants (FIG. 3M), supporting an important role for these Tasi-ARFs in SAR. Notably, Tasi-ARF conferred SAR on ago7 and tas3a plants was weaker than that in Col-0 (FIG. 3M). Considering both ago7 and tas3a mutants lack all Tasi-ARFs generated from Tas3a₅₅₅ transcript, a D7/D8-mediated partial SAR on these mutants suggests a role for other Tasi-ARFs in SAR.

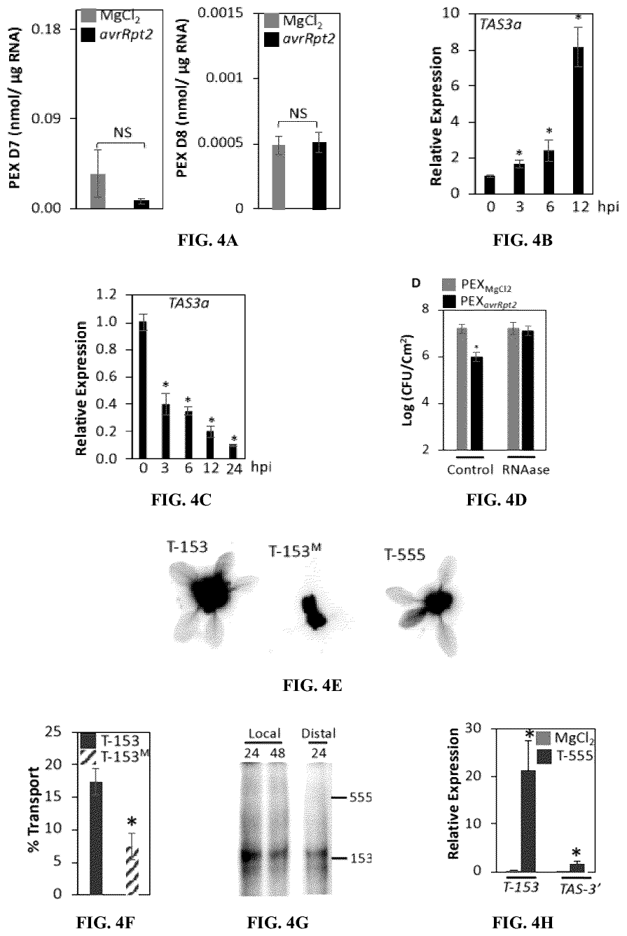

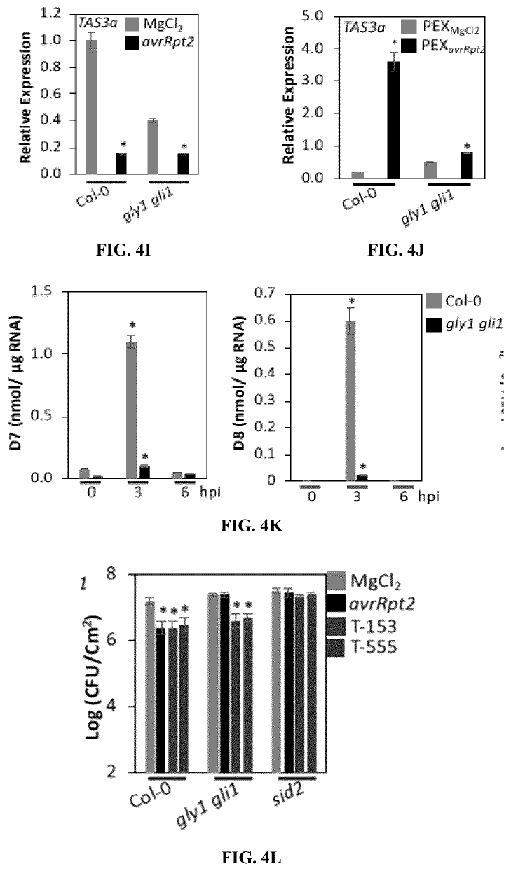

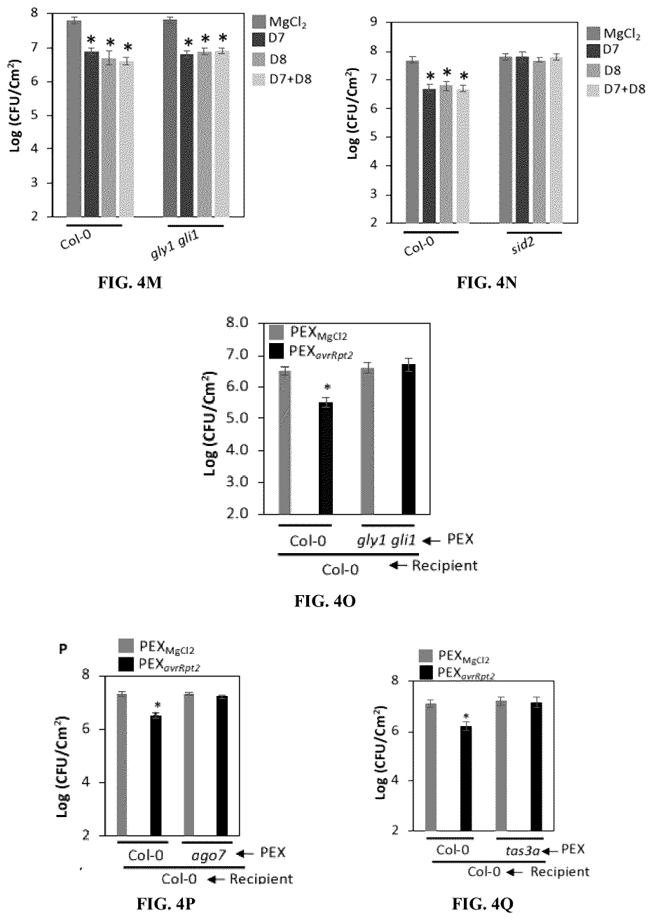

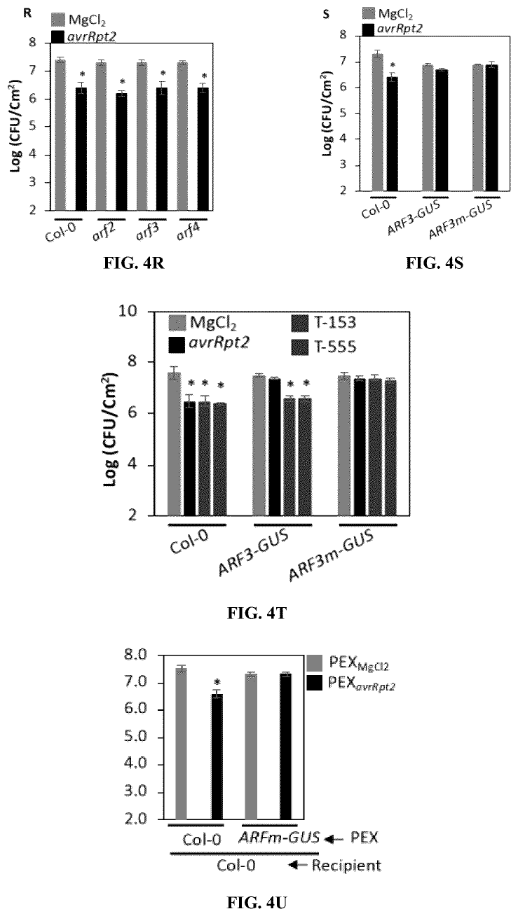

Since localized application of TAS3a and Tasi-ARFs D1 and D8 was able to induce SAR, it was possible that these RNA molecules might be mobile. Interestingly, although both D7 and D8 Tasi-ARFs were present in the PEX collected after 3 or 12 hpi, neither of these sRNAs were induced in PEXₐᵥᵣ (FIG. 4A, data shown for 3 hpi). In contrast, Tas3a transcript levels were significantly higher in PEXₐᵥᵣ, with transcript accumulating within 3 h and reaching highest levels by 12 h post pathogen inoculation (FIG. 4B). This correlated with the drastic reduction in TAS3a transcript levels in pathogen infected wild-type plants (FIG. 11A). A time-course analysis showed that TAS3a expression declined within 3 h post inoculation and levels were lowest at 12-24 h post inoculation (FIG. 4C), suggesting that the TAS3a transcript was rapidly transported away from the site of pathogen infection.

To determine if the presence of TAS3a in PEX, and thereby its transport via PEX was essential for SAR, the effect of RNAase treatment on PEX_{MgCl2} and PEXₐᵥᵣ was tested. Indeed, RNAase-treated PEXₐᵥᵣ (PEXₐᵥᵣ₋RNAase) was unable to induce SAR in wild-type plants (FIG. 4D), which in turn correlated with the absence of detectable TAS3a transcript in PEXₐᵥᵣ₋RNAase (FIG. 11B). In contrast, levels of SA and AzA in PEXₐᵥᵣ₋RNAase were comparable to those in PEXₐᵥᵣ (FIGS. 11C-D). The systemic transport of TAS3a was further confirmed by monitoring the movement of radiolabeled Tas3a₁₅₃ and full length Tas3a₅₅₅ transcripts. Approximately 12-17% of ³²P applied locally in the form of ³²P-rATP containing Tas3a₁₅₃ or the TAS3a₅₅₅, was detected in distal tissues (FIGS. 4E and 11E). This suggested that both transcripts were mobile. To determine if transport of the infiltrated transcripts occurred in a non-specific manner via apoplast, transport of the mutant TAS3a_{153M} RNA was evaluated. The TAS3a_{153M} RNA was significantly less amenable to systemic transport and was unable to efficiently spread throughout distal leaves (FIGS. 4E-F). This suggested that a significant percentage of TAS3a was transported via the symplast, which in turn correlated well with the drastically reduced SAR-inducing ability of Tas3a_{153M} (FIG. 3G). Interestingly, RNA gel analysis of plants infiltrated with ³²P-rATP labeled TAS3a₅₅₅ transcript showed that it was processed to greater than ˜>153 nt before translocation to the distal tissues (FIG. 4G). Likewise, plants infiltrated with cold Tas3a₅₅₅ transcript showed a higher percentage of 5′˜153 nt RNA compared to 3′ region (FIG. 4H).

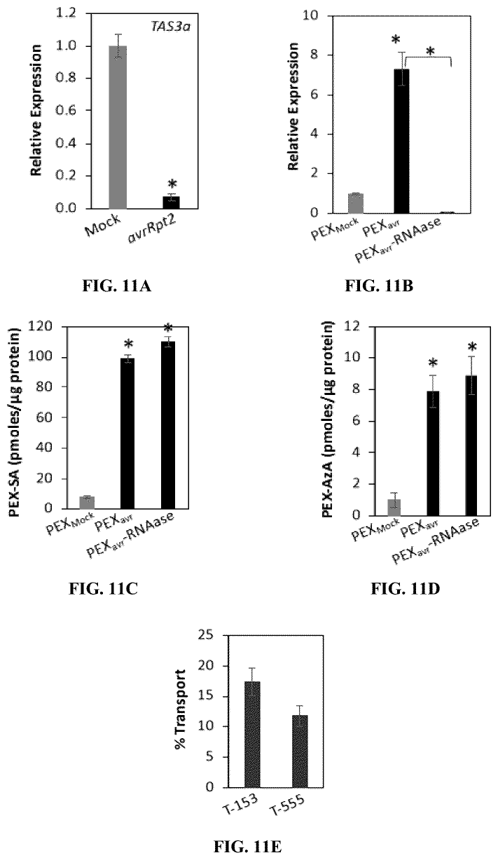

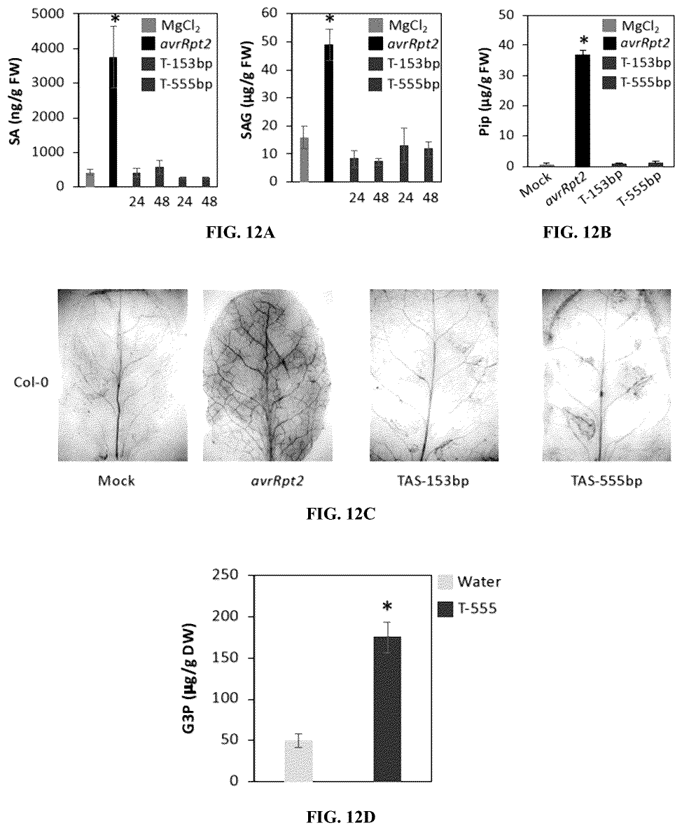

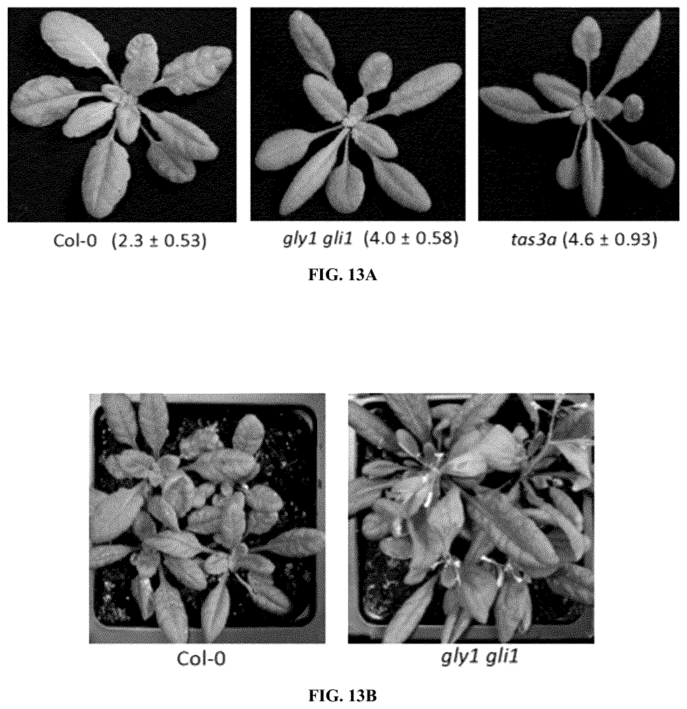

Although localized application of TAS3a transcript did not induce SA, Pip, or ROS accumulation (FIGS. 12A-C), they did increase G3P levels (FIG. 12D), suggesting a link between G3P and TAS3a in the SAR pathway. To test this association further, TAS3a levels were first assayed in gly1 gli1 plants, which are defective in G3P biosynthesis. Basal levels of the TAS3a transcript were significantly reduced in gly1 gli1 plants (FIG. 4I). TAS3a levels were also reduced in PEXₐᵥᵣ from gly1 gli1 plants (FIG. 4J). This in turn was consistent with lower levels of Tasi-ARFs (FIG. 4K), and the associated zippy and early flowering phenotypes displayed by gly1 gli1 plants (FIGS. 13A-B). This led to the possibility that impaired SAR in gly1 gli1 plants was due to reduced levels of TAS3a and/or Tasi-ARFs. Indeed, localized application of Tas3a₁₅₃, Tas3a₅₅₅ or Tasi-ARFs D7 or D8 conferred robust SAR on gly1 gli1 plants (FIGS. 4L-M). Together, these results suggested that defective SAR in gly1 gli1 plants was associated with reduced levels of Tasi-ARFs. Unlike gly1 gli1, localized application of Tas3a₁₅₃, Tas3a₅₅₅ or Tasi-ARFs D7 or D8 did not confer SAR on the SA deficient sid2 plants (FIGS. 4L and 4N).

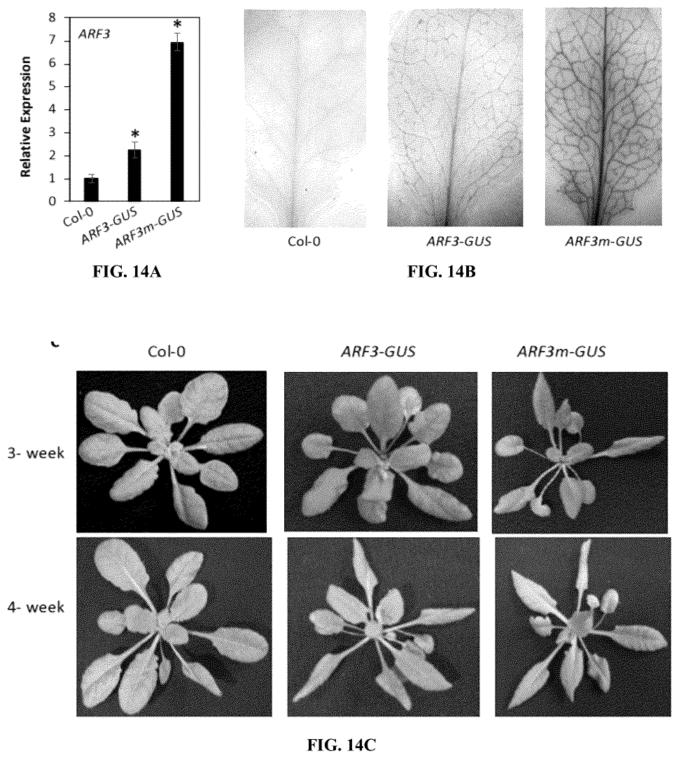

Notably, the G3P-deficient plants were unable to generate the SAR associated mobile signal; PEX collected from pathogen (Pst avrRpt2)-infected gly1 gli1 plants (PEXₐᵥᵣ) was unable to induce SAR on Col-0 plants (FIG. 4O). Likewise, PEXₐᵥᵣ from tas3a or ago7 was unable to confer SAR on Col-0 plants, suggesting that these too were impaired in the generation of the mobile signal (FIGS. 4P-Q). A common phenotype shared between gly1 gli1, tas3a and ago7 was that they all lacked Tasi-ARFs. In view thereof, a role of TAS3a target genes ARF 2, 3 and 4 in SAR was tested. SAR was first assayed in plants with knockout (KO) mutations in ARF2, 3, or 4. The ARF KO plants induced normal SAR (FIG. 4R). However, transgenic plants (expressing ARF3-GUS under the ARF3 native promoter in wild-type background) expressing increased ARF3 (FIGS. 14A-B) exhibited the zippy phenotype (FIG. 14C) and were compromised for SAR (FIG. 4S). Moreover, the zippy phenotype and ARF3 levels were more pronounced in plants expressing a mutant form of ARF3 (designated ARF3m) that is uncleavable by Tasi-ARFs (FIGS. 14A-C). As expected, the ARF3m-GUS plants showed compromised SAR (FIG. 4S).

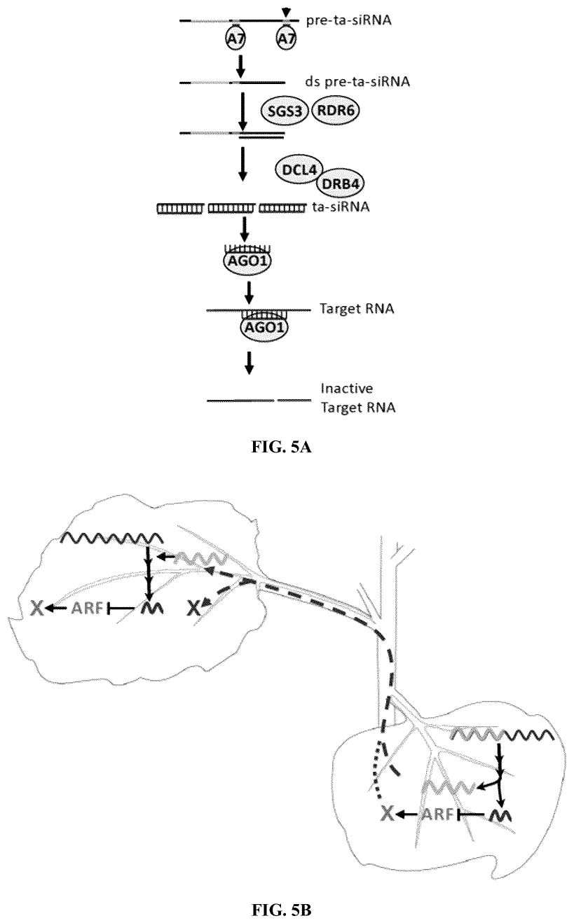

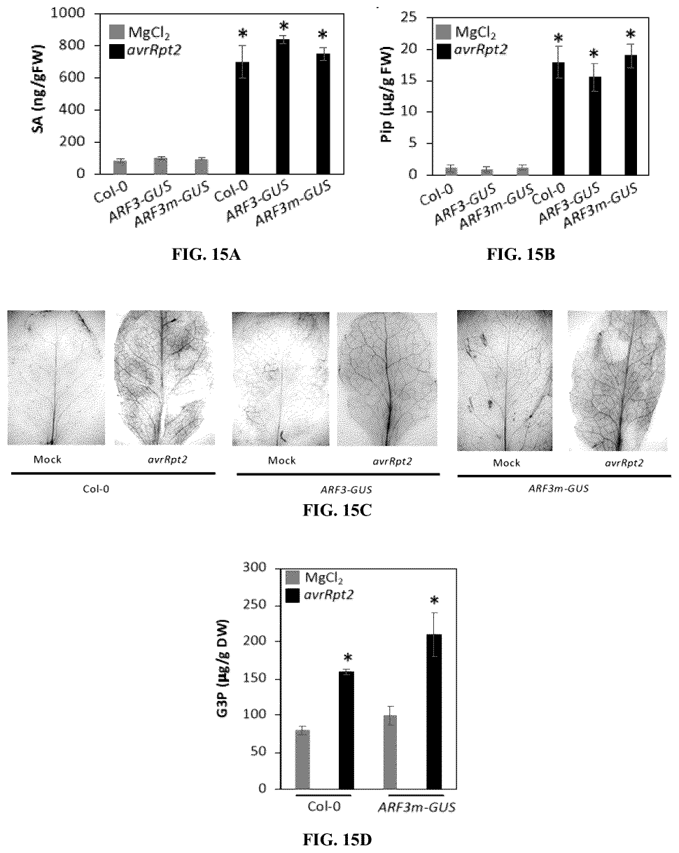

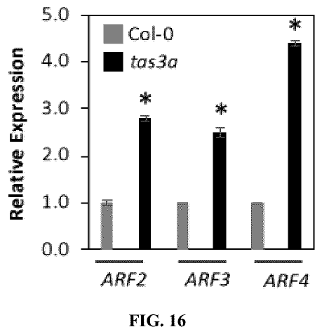

Since normal levels of SA, Pip, ROS and G3P in ARF3m-GUS plants suggests that increased ARF3 does not affect SAR by altering any of the known SAR chemical signals (FIGS. 15A-D), the ability of TAS3a to induce SAR was next evaluated in plants expressing ARF3-GUS and ARF3m-GUS. Localized application of either Tas3a₁₅₃, or Tas3a₅₅₅ transcripts was able to restore SAR in ARF3-GUS plants, but not ARF3m-GUS plants (FIG. 4T). These results suggest that a threshold level of TAS3a, and thereby Tasi-ARFs, are required to downregulate ARF3 expression, and are consistent with upregulation of ARF2, 3, 4 in tas3a plants (FIG. 16. Thus, TAS3a functions by negatively regulating ARF expression, which, without wishing to be limited by theory, is believed to negatively regulate a positive regulator (designated as X in FIG. 5A-B). The gly1 gli1, ago7 and tas3a plants lack Tasi-ARFs and thereby are unable to repress ARF genes, and relieve ARF-mediated suppression of X. Consistent with this notion, PEXₐᵥᵣ from ARF3m-GUS plants was unable to confer SAR on wild-type plants (FIG. 4U).

Without wishing to be bound by theory, it is believed that Tasi-ARFs-mediated repression of ARF3, and possibly that of ARF2 and ARF4, is required for generation of X, which initiates SAR in distal issues (FIG. 5A-B). The ago7 plants produce normal levels of Tas3a but are unable to generate Tasi-ARFs and their SAR defect is associated with lack of Tasi-ARFs. Likewise, SAR defect in both tas3a and gly gli1 plants can be complemented by Tasi-ARFs, suggesting an important role for Tasi-ARFs in SAR. Plants overexpressing ARF3 contain normal levels of Tasi-ARFs but remain SAR compromised since Tasi-ARFs are unable to target ARF3. Although Tasi-ARFs were induced upon pathogen infection, the PEX levels of Tasi-ARFs did not increase in response to pathogen. Notably, in addition to generating Tasi-ARFs, TAS3a was processed into ˜200-nt truncated transcript, which was rapidly transported to distal tissues. The Tas3a₁₅₃ transcript is an important accessory for SAR and TAS3a encodes a protein which has been suggested to facilitate generation of Tasi-ARFs. These results suggest that transport of 3′ truncated TAS3a facilitates generation of Tasi-ARFs in the distal tissues. The conserved nature of TAS3a-mediated regulation of ARFs in plants supports the abilities of Arabidopsis and soybean PEX to confer resistance in monocots and dicots.

### Materials and Methods

Plant growth conditions and genetic analysis—Plants were grown in MTPS 144 Conviron (Winnipeg, MB, Canada) walk-in chambers at 22° C., 65% relative humidity and 14 h light and 10 h dark photoperiod. These chambers were equipped with cool white fluorescent bulbs (Sylvania, FO96/841/XP/ECO). The photon flux density (PFD) of the day period was 106.9 μmoles m⁻² s⁻¹ (measured using a digital light meter, Phytotronic Inc, MO). Plants were grown on autoclaved Pro-Mix soil (Premier Horticulture Inc., PA, USA). Soil was fertilized once using Scotts Peter's 20:10:20 peat lite special general fertilizer that contained 8.1% ammoniacal nitrogen and 11.9% nitrate nitrogen (Scottspro.com). Plants were irrigated using deionized or tap water. The tas3a (GK-621G08) and tas3b (GK-649H12) plants used in this study are described earlier. The tas2 homozygous plants were identified from SALK insertion line (014168) obtained from the ABRC database. The ago1-27 hypomorphic mutants were described previously. The ago7, sgs3 and rdr6 seeds were obtained from the Arabidopsis database. The gly1 gli1 double mutant plants were generated by crossing gly1-1 with gli1-1 and both these genotypes were described previously.

Generation of DRB overexpressing plants—For transgenic overexpression of DRBs, the cDNA spanning the coding region were cloned into pGWB2 vector, which after confirmation of the DNA sequence was transformed into Col-0 plants. The transgenic plants were selected on plates containing kanamycin (50 μg/ml) and hygromycin (17 μg/ml).

RNA extraction, quantitative real-time PCR, and in vitro transcription—Small-scale extraction of RNA from two or three leaves (per sample) was performed with the TRIzol reagent (Invitrogen, CA), following the manufacturer's instructions. RNA quality and concentration were determined by gel electrophoresis and determination of A₂₆₀. Reverse transcription (RT) and first strand cDNA synthesis were carried out using Superscript II (Invitrogen, CA). Quantitative RT-PCR was carried out as described before. Each sample was run in triplicates and ACTINII (At3g18780) or UBC2 expression levels were used as internal control for normalization. Cycle threshold values were calculated by SDS 2.3 software.

The synthesis of TAS3a RNA was carried out by in vitro transcription using T7 RNA polymerase. The TAS3a sequences were cloned in the pBluescript-SK²⁺ vector, which after confirmation of the DNA sequence were linearized and transcribed. The in vitro synthesized transcripts were analyzed by RNA gel electrophoresis, purified, quantified using nanodrop and used for SAR assays. Radiolabeled transcripts were synthesized by replacing ATP with ³²P-ATP during transcription reaction.

Protein extraction and immunoblot analysis—Proteins were extracted in buffer containing 50 mM Tris-HCl, pH 7.5, 10% glycerol, 150 mM NaCl, 10 mM MgCl₂, 5 mM EDTA, 5 mM DTT, and 1× protease inhibitor cocktail (Sigma-Aldrich, St. Louis, MO). Protein concentration was measured by the Bio-RAD protein assay (Bio-Rad, CA). For Ponceau-S staining, PVDF membranes were incubated in Ponceau-S solution (40% methanol (v/v), 15% acetic acid (v/v), 0.25% Ponceau-S). The membranes were destained using deionized water. Proteins (˜150 μg) were fractionated on a 12-15% SDS-PAGE gel and subjected to immunoblot analysis using ∝-TAS-50aa or ∝-DRB antibodies. The ∝-TAS-50aa was raised in rabbits using an in vitro synthesized peptide (Pepmic Co. Ltd, China). The DRB1 and DRB4 antibodies have been described earlier. Immunoblots were developed using ECL detection kit (Roche) or alkaline phosphatase-based color detection.

Pathogen infection and collection of phloem exudate—Inoculations with Pseudomonas syringae DC 3000 were conducted as described before. The bacterial cultures were grown overnight in King's B medium containing rifampicin and/or kanamycin. For analysis of SAR, the primary leaves were inoculated with MgCl₂ or the avr bacteria (10⁷ cfu ml⁻¹) and, 48 h later, the systemic leaves were inoculated with vir bacteria (10⁵ cfu ml⁻¹). Unless noted otherwise, samples from the systemic leaves were harvested at 3 dpi. Petiole exudates were collected in diethyl pyrocarbonate (DEPC) treated water as described earlier. PEX was collected for 3-48 and assayed for bacterial growth to ensure that it did not contain any viable bacteria. PEX RNA was extracted using the TRIzol reagent, quantified using nanodrop and cDNA synthesized from PEX RNA was evaluated for contamination with leaf RNA by assaying for amplification of Rubisco genes. Each sample was run in triplicates and UBC9 expression levels were used as internal control for normalization. Cycle threshold values were calculated by SDS 2.3 software.

Chemical and RNA treatments—SA, G3P, AzA, and Pip treatments were carried out by using 500 μM, 100 μM, 1000 μM, and 1000 μM solutions, respectively. TAS3a RNA was suspended at a concentration of 0.0075-75 ng/μl of DEPC water and ˜40 μl was infiltrated per leaf. AzA was prepared in methanol and diluted in water. SA, G3P and Pip were prepared and diluted in water. All dilutions were freshly prepared prior to performing biological experiments.

G3P, SA, and Pip quantifications—G3P quantifications were carried out as described earlier. SA and SA glucoside (SAG) were extracted and measured from ˜ 0.1 g of fresh weight leaf tissue, as described before. Pip quantifications were carried out using gas chromatography (GC)-mass spectrometry (MS). For quantification of SA and AzA in PEX, the samples were dried under nitrogen, suspended in acetonitrile and derivatized with N-methyl-N-(tert-butyldimethylsilyl) trifluoroacetamide (MTBSTFA) containing 1% tert-butyldimethylchlorosilane (TBDMCS) and analysed by GC-MS.

TAS3a transport assays—For TAS3a transport, [³²P] labelled TAS3a RNAs were synthesized by in vitro transcription (1 μCi/ml, specific activity 38 mCi/mmol; Perkin Elmer Inc.) and the purified RNAs were suspended in DEPC water and used for infiltrations. The resulting solution contained 22.9 pM of 555 bp and 142.9 pM of 153 bp TAS3a transcripts and was injected into abaxial surface of four-week-old Arabidopsis leaves. Three leaves per plant were infiltrated with ˜0.04 ml of ³²P-TAS3a transcripts. The plants were then kept in a growth chamber set at 14 h light and 10 h dark photoperiods. The leaf samples were extracted using RNA extraction method described above. The samples were quantified using a liquid scintillation counter and extracts containing [³²P] radioactivity were loaded onto a silica gel 60 thin layer chromatography (TLC) plate and developed using butanol:acetic acid:water (3:1:1, by vol). The TLC plates were exposed in a storage phosphorimage screen (GE) and the bands were visualized by Typhoon PhosphorImager.

RNA sequencing—Sequencing libraries were constructed and Illumina paired-end (PE) sequencing was performed using the Hiseq2000 platform at Beijing Yuanquanyike Biotech, Beijing, China, according to the manufacturer's instructions (Illumina, San Diego, CA). All of the raw reads were filtered to exclude reads that failed the built-in Failed Chastity Filter in the Illumina software according to the relation “failed-chastity≤1,” using a chastity threshold of 0.6, on the first 25 cycles. Likewise, reads with adaptor contamination were discarded, low-quality reads were masked with ambiguous sequences “N” and reads with more than 10% Q<20 were removed. All the filtered reads were de novo assembled using Trinity (RRID: SCR_013048, ver. trinityrnaseq_r2013_08_14) with paired-end method and default parameters as previous study on optimal assembly strategy.

Confocal microscopy—For confocal imaging, samples were scanned on an Olympus FV1000 microscope (Olympus America, Melvile, NY). GFP was excited using 488 nm laser line. Water-mounted sections of leaf tissue were examined by confocal microscopy using a water immersion PLAPO60XWLSM 2 (NA 1.0) objective on a FV1000 point-scanning/point-detection laser scanning confocal microscope (Olympus) equipped with lasers spanning the spectral range of 405-633 nm. GFP images (40× magnification) were acquired at a scan rate of 10 ms/pixel. Olympus FLUOVIEW 1.5 was used to control the microscope, image acquisition and the export of TIFF files.

Statistics and reproducibility—For pathogen assays, ˜16 plants/genotype/treatment were analyzed in a single experiment. At least 3-4 technical replicates/genotype/treatment were plated. For metabolite quantification, ˜12 plants/genotype/treatment were analyzed in each experiment. Experiments were repeated at least two-three times with a different set of plants as indicated in the figure legends. Unless otherwise mentioned error bars indicate SD.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Wang, C. et al. Free radicals mediate systemic acquired resistance. Cell Reports 7, 348-355, doi: 10.1016/j.celrep.2014.03.032 (2014).
2. Wang, C. et al. Pipecolic acid confers systemic immunity by regulating free radicals. Science Advances 4, eaar4509, doi: 10.1126/sciadv.aar4509 (2018).
3. Wendehenne, D., Gao, Q.-m., Kachroo, A. & Kachroo, P. Free radical-mediated systemic immunity in plants. Current Opinion in Plant Biology 20, 127-134, doi: dx.doi.org/10.1016/j.pbi.2014.05.012 (2014).
4. Singh, A., Lim, G. H. & Kachroo, P. Transport of chemical signals in systemic acquired resistance. Journal of Integrative Plant Biology 59, 336-344, doi: doi: 10.1111/jipb.12537 (2017).
5. Shine, M. B., Xiao, X., Kachroo, P. & Kachroo, A. Signaling mechanisms underlying systemic acquired resistance to microbial pathogens. Plant Science 279, 81-86, doi: doi.org/10.1016/j.plantsci.2018.01.001 (2018).
6. Vlot, A. C., Dempsey, D. M. A. & Klessig, D. F. Salicylic acid, a multifaceted hormone to combat disease. Annual Review of Phytopathology 47, 177-206, doi: 10.1146/annurev.phyto.050908.135202 (2009).
7. Park, S.-W., Kaimoyo, E., Kumar, D., Mosher, S. & Klessig, D. F. Methyl salicylate is a critical mobile signal for plant systemic acquired resistance. Science 318, 113-116, doi: 10.1126/science.1147113 (2007).
8. Návarová, H., Bernsdorff, F., Doring, A.-C. & Zeier, J. Pipecolic acid, an endogenous mediator of defense amplification and priming, is a critical regulator of inducible plant immunity. The Plant Cell 24, 5123-5141, doi: 10.1105/tpc. 112.103564 (2012).
9. Jung, H. W., Tschaplinski, T. J., Wang, L., Glazebrook, J. & Greenberg, J. T. Priming in systemic plant immunity. Science 324, 89-91, doi: 10.1126/science. 1170025 (2009).
10. Yu, K. et al. A feedback regulatory loop between G3P and lipid transfer proteins DIR1 and AZI1 mediates azelaic-acid-induced systemic immunity. Cell Reports 3, 1266-1278, doi: 10.1016/j.celrep.2013.03.030 (2013).
11. Gao, Q.-m. et al. Mono- and digalactosyldiacylglycerol lipids function nonredundantly to regulate systemic acquired resistance in plants. Cell Reports 9, 1681-1691, doi: 10.1016/j.celrep.2014.10.069 (2014).
12. Chanda, B. et al. Glycerol-3-phosphate is a critical mobile inducer of systemic immunity in plants. Nature Genetics 43, 421-427, doi: www.nature.com/ng/journal/v43/n5/abs/ng.798.html#supplementary-information (2011).
13. D. Wendehenne, J. Durner, D. F. Klessig, Nitric oxide: a new player in plant signalling and defence responses. Current Opinion in Plant Biology 7, 449-455 (2004).
14. Lenk, M. et al. Pipecolic acid is induced in barley upon infection and triggers immune responses associated with elevated nitric oxide accumulation. Molecular Plant-Microbe Interactions (2019).
15. Lim, G.-H. et al. Plasmodesmata localizing proteins regulate transport and signaling during systemic acquired immunity in plants. Cell Host & Microbe 19, 541-549, doi: 10.1016/j.chom.2016.03.006 (2016).
16. Wenig, M. et al. Systemic acquired resistance networks amplify airborne defense cues. Nature communications 10, 1-14 (2019).
17. Bernsdorff, F. et al. Pipecolic acid orchestrates plant systemic acquired resistance and defense priming via salicylic acid-dependent and-independent pathways. The Plant Cell 28, 102-129 (2016).
18. Lim, G.-H. et al. The plant cuticle regulates apoplastic transport of salicylic acid during systemic acquired resistance. Science Advances 6, eaaz0478 (2020).
19. Lim, G.-H. et al. The analogous and opposing roles of double-stranded RNA-binding proteins in bacterial resistance. Journal of experimental botany 70, 1627-1638 (2019).
20. Zhu, S. et al. Double-stranded RNA-binding protein 4 is required for resistance signaling against viral and bacterial pathogens. Cell reports 4, doi: 10.1016/j.celrep.2013.08.018 (2013).
21. Pelissier, T. et al. Double-stranded RNA binding proteins DRB2 and DRB4 have an antagonistic impact on polymerase IV-dependent siRNA levels in Arabidopsis. RNA 17, doi: 10.1261/rna.2680711 (2011).
22. Lim, G.-H. et al. COP1, a negative regulator of photomorphogenesis, positively regulates plant disease resistance via double-stranded RNA binding proteins. PLOS Pathogens 14, e1006894, doi: 10.1371/journal.ppat. 1006894 (2018).
23. Wang, M.-B., Masuta, C., Smith, N. A. & Shimura, H. RNA silencing and plant viral diseases. Molecular Plant-Microbe Interactions 25, 1275-1285, doi: 10.1094/MPMI-04-12-0093-CR (2012).
24. Adenot, X. et al. DRB4-dependent TAS3 trans-acting siRNAs control leaf morphology through AGO7. Current Biology 16, 927-932, doi: 10.1016/j.cub.2006.03.035 (2006).
25. Montgomery, T. A. et al. Specificity of ARGONAUTE7-miR390 interaction and dual functionality in TAS3 trans-acting siRNA formation. Cell 133, 128-141 (2008).
26. de Felippes, F. F., Marchais, A., Sarazin, A., Oberlin, S. & Voinnet, O. A single miR390 targeting event is sufficient for triggering TAS3-tasiRNA biogenesis in Arabidopsis. Nucleic acids research 45, 5539-5554 (2017).
27. Marin, E. et al. miR390, Arabidopsis TAS3 tasiRNAs, and their AUXIN RESPONSE FACTOR targets define an autoregulatory network quantitatively regulating lateral root growth. The Plant Cell 22, 1104-1117 (2010).
28. Bazin, J. et al. Global analysis of ribosome-associated noncoding RNAs unveils new modes of translational regulation. Proceedings of the National Academy of Sciences 114, E10018-E10027 (2017).
29. Hou, C. Y. et al. Global analysis of truncated RNA ends reveals new insights into ribosome stalling in plants. The Plant Cell 28, 2398-2416 (2016).
30. Akulich, K. A. et al. Four translation initiation pathways employed by the leaderless mRNA in eukaryotes. Scientific reports 6, 37905 (2016).
31. Chaturvedi, R. et al. Plastid ω3-fatty acid desaturase-dependent accumulation of a systemic acquired resistance inducing activity in petiole exudates of Arabidopsis thaliana is independent of jasmonic acid. The Plant Journal 54, 106-117 (2008).
32. Shine, M. et al. Glycerol-3-phosphate mediates rhizobia-induced systemic signaling in soybean. Nature communications 10, 1-13 (2019).
33. Axtell, M. J., Snyder, J. A. & Bartel, D. P. Common functions for diverse small RNAs of land plants. The Plant Cell 19, 1750-1769 (2007).
34. Arikit, S. et al. An atlas of soybean small RNAs identifies phased siRNAs from hundreds of coding genes. The Plant Cell 26, 4584-4601 (2014).
35. Rajeswaran, R. & Pooggin, M. M. RDR6-mediated synthesis of complementary RNA is terminated by miRNA stably bound to template RNA. Nucleic Acids Research 40, 594-599, doi: 10.1093/nar/gkr760 (2011).
36. Wu, X. et al. Genome-wide landscape of polyadenylation in Arabidopsis provides evidence for extensive alternative polyadenylation. Proceedings of the National Academy of Sciences 108, 12533-12538 (2011).
37. Su, Z. et al., The THO complex non-cell-autonomously represses female germline specification through the TAS3-ARF3 module. Current Biology 27, 1597-1609. e1592 (2017).
38. Morel, J.-B. et al., Fertile hypomorphic ARGONAUTE (ago1) mutants impaired in post-transcriptional gene silencing and virus resistance. The Plant Cell 14, 629-639 (2002).
39. Mandal, M. K. et al., Oleic acid-dependent modulation of NITRIC OXIDE ASSOCIATED1 protein levels regulates nitric oxide-mediated defense signaling in Arabidopsis. The Plant Cell 24, 1654-1674 (2012).
40. Zhang, D.-X. et al., Regulation of a chemical defense against herbivory produced by symbiotic fungi in grass plants. Plant Physiology 150, 1072-1082 (2009).
41. Xia, Y. et al., An intact cuticle in distal tissues is essential for the induction of systemic acquired resistance in plants. Cell Host & Microbe 5, 151-165 (2009).
42. Tetyuk, O. et al., Collection and analysis of Arabidopsis phloem exudates using the EDTA-facilitated method. JoVE (Journal of Visualized Experiments), e51111 (2013).
43. Chandra-Shekara, A. C. et al., Light-dependent hypersensitive response and resistance signaling against Turnip Crinkle Virus in Arabidopsis. Plant Journal 45, 320-334 (2006).
44. He, B. et al., Optimal assembly strategies of transcriptome related to ploidies of eukaryotic organisms. BMC genomics 16, 65 (2015).
45. Lee, J.-Y. et al., A plasmodesmata-localized protein mediates crosstalk between cell-to-cell communication and innate immunity in Arabidopsis. The Plant Cell 23, 3353-3373 (2011).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

What is claimed is:

1. A method of conferring systemic acquired resistance (SAR) to a plant, comprising delivering to a plant an exogenous trans-acting small interfering RNA3a (Tas3a) RNA, wherein the plant expresses a full-length Tas3a RNA transcript, and wherein the exogenous Tas3a RNA comprises one or more of SEQ ID NOs: 2, 3, 4, 8 and 10.

2. The method of claim 1, wherein the full-length Tas3a RNA transcript is expressed from the exogenous Tas3a RNA.

3. The method of claim 1, wherein the exogenous Tas3a RNA comprises SEQ ID NO:2.

4. The method of claim 1, wherein the exogenous Tas3a RNA comprises SEQ ID NO:2 and one or more of SEQ ID NO: 3, 4, 8 and 10.

5. The method of claim 1, wherein the full-length Tas3a RNA transcript is expressed from the wildtype plant genome.

6. The method of claim 1, wherein the sequence of the full-length Tas3a RNA transcript comprises SEQ ID NO: 2.

7. The method of claim 5, wherein the exogenous Tas3a RNA is SEQ ID NO:3 or SEQ ID NO:4.

8. The method of claim 5, wherein the exogenous Tas3a RNA is SEQ ID NO:8 or SEQ ID NO:10.

9. The method of claim 1, wherein delivery comprises localized application of the exogenous Tas3a RNA to the plant.

10. The method of claim 8, wherein localized application comprises injection or infiltration into a tissue of the plant.

11. The method of claim 8, wherein the systemic acquired resistance (SAR) comprises a response distal to the site of localized application.

12. The method of claim 1, wherein the systemic acquired resistance (SAR) confers resistance against a pathogen.

13. A method of conferring systemic acquired resistance (SAR) to a plant, comprising delivering an exogenous trans-acting small interfering RNA3a (Tas3a) RNA to an SAR deficient or SAR-compromised plant, wherein the exogenous Tas3a RNA comprises one or more of SEQ ID NOs: 2, 3, or 4, and wherein application of the exogenous Tas3a RNA confers SAR to the plant.

14. The method of claim 13, wherein delivery comprises localized application of the exogenous Tas3a RNA to the plant.

15. The method of claim 13, wherein localized application comprises injection or infiltration into a tissue of the plant.

16. The method of claim 13, wherein the SAR comprises a response distal to the site of localized application.

17. The method of claim 13, wherein the SAR confers resistance against a pathogen.

18. The method of claim 13, wherein the exogenous Tas3a RNA comprises SEQ ID NO:2.

19. The method of claim 18, wherein the method further comprising delivering a second exogenous Tas3a RNA and wherein the second exogenous Tas3a RNA comprises any one of SEQ ID Nos: 3, 4, 8 and 10.


SEQUENCE LISTING (11 sequences)

SEQ ID NO: 1 - DNA, 555 bp, Arabidopsis thaliana
  misc_feature 58..84: note = open reading frame
  misc_feature 85..210: note = open reading frame
  misc_binding 217..239: bound_moiety = miR390-AG07 targeting site; note = miR390-AGO7 targeting site
  misc_binding 471..480: bound_moiety = miR390-AG07 targeting site; note = miR390-AGO7 targeting site
  source 1..555: mol_type = other DNA
atcccaccgtttcttaagactctctctctttctgttttctatttctctctctctcaaatgaaagagagagaagagctcccatggatgaaattagcgagaccgaagtttctccaaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 2 - RNA, 555 nt, Arabidopsis thaliana
  source 1..555: mol_type = other RNA
atcccaccgtttcttaagactctctctctttctgttttctatttctctctctctcaaatgaaagagagagaagagctcccatggatgaaattagcgagaccgaagtttctccaaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 3 - RNA, 21 nt, Arabidopsis thaliana
  source 1..21: mol_type = other RNA
tcttgaccttgtaaggccttt

SEQ ID NO: 4 - RNA, 21 nt, Arabidopsis thaliana
  source 1..21: mol_type = other RNA
tcttgaccttgtaagacccca

SEQ ID NO: 5 - DNA, 126 bp, Arabidopsis thaliana
  source 1..126: mol_type = other DNA
atgaaattagcgagaccgaagtttctccaaggcattaaggaaaacataacctccgtgatgcatagagattattggatccgctgtgctgagacattgagtttttcttcggcattccagtttcaatga

SEQ ID NO: 6 - RNA, 126 nt, Arabidopsis thaliana
  source 1..126: mol_type = other RNA
atgaaattagcgagaccgaagtttctccaaggcattaaggaaaacataacctccgtgatgcatagagattattggatccgctgtgctgagacattgagtttttcttcggcattccagtttcaatga

SEQ ID NO: 7 - DNA, 153 bp, Arabidopsis thaliana
  source 1..153: mol_type = other DNA
atgaaagagagagaagagctcccatggatgaaattagcgagaccgaagtttctccaaggcattaaggaaaacataacctccgtgatgcatagagattattggatccgctgtgctgagacattgagtttttcttcggcattccagtttcaatga

SEQ ID NO: 8 - RNA, 153 nt, Arabidopsis thaliana
  source 1..153: mol_type = other RNA
atgaaagagagagaagagctcccatggatgaaattagcgagaccgaagtttctccaaggcattaaggaaaacataacctccgtgatgcatagagattattggatccgctgtgctgagacattgagtttttcttcggcattccagtttcaatga

SEQ ID NO: 9 - DNA, 345 bp, Arabidopsis thaliana
  source 1..345: mol_type = other DNA
taaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 10 - RNA, 345 nt, Arabidopsis thaliana
  source 1..345: mol_type = other RNA
taaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 11 - protein, 50 aa, Arabidopsis thaliana
MKEREELPWMKLARPKFLQGIKENITSVMHRDYWIRCAETLSFSSAFQFQ